(19) United States
(12) Patent Application Publication
(10) Pub. No.: US 2026/0275281 A1
(43) Pub. Date: Sep. 17, 2026

(54) COMPOSITIONS AND METHODS FOR DIFFERENTIATING RPE CELLS

(71) Applicant: STEMCELL TECHNOLOGIES CANADA INC., Vancouver (CA)

(72) Inventors: Ishpreet DHILLON, Vancouver (CA); Valentina MARCHETTI, Vancouver (CA); Li Jing SHEAVES, Vancouver (CA)

(21) Appl. No.: 19/469,669

(22) PCT Filed: Apr. 2, 2024
(86) PCT No.: PCT/CA2024/050420

§ 371 Date: Sep. 26, 2025

Related U.S. Application Data
(60) Provisional application No. 63/457,430, filed on Apr. 6, 2023.

(51) Int. Cl. C12N 5/079

(52) U.S. Cl. CPC C12N 5/0621; C12N 2501/155; C12N 2501/16; C12N 2501/415; C12N 2506/45

(57) ABSTRACT

The present disclosure relates to applications of culturing retinal pigment epithelium (RPE) cells. The method comprises a) providing a population of PSC; b) exposing the population of PSC to a first culture environment comprising an extracellular matrix protein and an inhibitor of BMP signaling; c) culturing the population of PSC in the first culture environment for between about 0 to 60 hours; and d) deriving the population of cells fated for the retinal linage.

### CROSS-REFERENCE TO RELATED APPLICATION

This application claims the benefit of U.S. Provisional Patent Application No. 63/457,430, filed Apr. 6, 2023, the entire contents of which is hereby incorporated by reference in its entirety.

### TECHNICAL FIELD

This disclosure relates to cell culture applications, and more specifically to cell differentiation and/or maturation applications. Still more specifically, this disclosure relates to culturing retinal pigment epithelium (RPE) cells and/or progenitors thereof.

### BACKGROUND

The retina is the inner layer of the eye and is a complex, layered structure of neurons that capture and process light: light signals are converted into electrical signals that travel through the optic nerve to the visual center of the brain.

The Retinal Pigment Epithelium (RPE) is a monolayer of cells which is located at the outermost layer of the retina. The basal side of the RPE is connected to Bruch's membrane and the choroid, whereas the apical side is connected to the outer segment of photoreceptor cells through microvilli structures extending from RPE cells. RPE cells are a highly phagocytic cell type and are able to internalize photoreceptor outer segments (POS) from overlying rod or cone photoreceptor cells. The RPE performs several critical functions in vision such as light absorption, formation of the outer blood-retinal barrier, nutrient and ion transport, retinoid cycling, phagocytosis of spent photoreceptor outer segments and growth factor secretion (Boulton et al. (2001) Eye 15, 384-389). The RPE has a dark brown color owing to its melanin content, which reduces damage to the retina and internal nerves from ultraviolet light.

RPE structure and function are essential to normal vision, and changes in the RPE can impair function and lead to degenerative retinal diseases such as age-related macular degeneration (AMD), retinitis pigmentosa (RP) and Stargardt disease (SD). AMD is the leading cause of severe vision loss in adults over 60 years of age and in 2020 was estimated to affect ~196 million people worldwide. RP causes visual loss in childhood or young adulthood and affects 100,000 people in the United States. SD has a prevalence rate of 1 in 10,000 births and is the most common form of inherited juvenile macular degeneration (Yang S et al. (2021) Front Pharmacol. 12:727870). There are currently no therapies for these degenerative disorders. Therefore, understanding the development of RPE in vitro may help elucidate pathogenesis of related conditions or diseases, and may aid development of new therapeutic approaches.

RPE cells produced in vitro can be used to study RPE development, to identify factors that cause damage to the RPE, or to identify agents that can be used to promote repair of endogenous RPE cells. Furthermore, RPE cells generated in vitro can themselves be used for cellular therapy which would involve replacing or restoring all or a portion of a patient's damaged RPE cells.

Pluripotent stem cells (PSCs), which include both embryonic stem cells (ESCs) and induced pluripotent stem cells (iPSC), would be an ideal starting cell type for in vitro generation of RPE cells. Production of RPE from PSC may accelerate research to understand various eye diseases and can also provide an unlimited source of RPE for retinal cell therapies. Accordingly, there is a need for fast and efficient in vitro protocols to generate RPE from PSC, and more specifically to higher compliance protocols suitable for cell therapy applications.

### SUMMARY

In one aspect of this disclosure are provided methods of differentiating immature and/or mature RPE, such as from one or more pluripotent stem cell (PSC). In one embodiment, the differentiation of immature and/or mature retinal pigment epithelium (RPE) goes through various stages. Thus, in one aspect of this disclosure are provided methods of differentiating a population of cells fated for the retinal lineage, comprising providing a population of PSC and exposing the population of PSC to a first culture environment comprising an extracellular matrix protein and an inhibitor of bone morphogenetic protein (BMP) signaling.

In one embodiment, a concentration of the inhibitor of BMP signaling is about 1000 nM or less, about 750 nM or less, about 600 nM or less, about 500 nM or less, about 250 nM or less, about 100 nM or less, or about 50 nM or less.

In one embodiment, the inhibitor of BMP signaling is comprised in a first cell culture medium. In one embodiment, the first cell culture medium comprises a basal medium.

In one embodiment, the method may further comprise culturing the population of PSC in the first culture environment for between about 0 to 60 hours to derive the population of cells fated for the retinal linage.

In one embodiment, the first culture environment is free of one or both of i) a direct wnt signaling antagonist and ii) an agonist of activin signaling.

In one embodiment, the extracellular matrix protein is coated on a culture surface. In one embodiment, the extracellular matrix protein is vitronectin, laminin, fibronectin, collagen, or a mixture comprising more than one of the foregoing. In one embodiment, the extracellular matrix protein is recombinant.

In one embodiment, methods of this disclosure may further comprise exposing the population of cells fated for the retinal lineage to a second culture environment and culturing the population of cells for between about 0 to 60 hours to derive a population of eye field progenitors.

In one embodiment, the second culture environment comprises the extracellular matrix protein and a second cell culture medium. In one embodiment, the second cell culture medium comprises IGF and a basal medium. In one embodiment, the second cell culture medium comprises an agonist of FGF signaling. In one embodiment, the second cell culture medium comprises a basal medium and one or more of IGF and an agonist of FGF signaling.

In one embodiment, methods of this disclosure may further comprise exposing the population of eye field progenitors to a third culture environment and culturing the population of eye field progenitors for between about 2 to 5 days to derive a population of optic vessel progenitors. In one embodiment, an optic vessel progenitor is bipotent for retinal pigment epithelium or neural retina.

In one embodiment, the third culture environment comprises the extracellular matrix protein and a third cell culture medium. In one embodiment, the third cell culture medium comprises IGF and a basal medium. In one embodiment, the third cell culture medium comprises an agonist of activin signaling. In one embodiment, the third cell culture medium comprises a basal medium and one or more of IGF and an agonist of activin signaling.

In one embodiment, methods of this disclosure may further comprise exposing the population of optic vessel progenitors to a fourth culture environment and culturing the population of optic vessel progenitors for between about 5 to 10 days to derive a population of immature retinal pigment epithelial cells.

In one embodiment, the fourth culture environment comprises the extracellular matrix protein and a fourth cell culture medium. In one embodiment, the fourth cell culture medium comprises a basal medium and one or more of an agonist of activin signaling, an agonist of wnt signaling, and an antagonist of FGF signaling.

In one embodiment, methods of this disclosure may further comprise dissociating the population of immature retinal pigment epithelial cells and replating the dissociated cells to enrich for immature retinal pigment epithelial cells. In one embodiment, the dissociated population of immature retinal pigment epithelial cells are not enriched through manual dissection, particle-based enrichment, or ligand-mediated enrichment.

In one embodiment, about 50% or more of the population of immature retinal pigment epithelial cells express PMEL17.

In one embodiment, the methods may further comprise maturing the population of immature retinal pigment epithelial cells for between about 1 to 5 weeks. In one embodiment, the population of immature retinal pigment epithelial cells are matured in contact with a maturation medium comprising a basal medium and at least a soluble source of iron. In one embodiment, the maturation medium further comprises one or more of a steroid hormone and a polyamine. In one embodiment, the maturation medium may further comprise progesterone and putrescine.

In one embodiment, about 80% or more of the matured RPE express RPE65.

In one embodiment, one or more of the first culture environment, the second culture environment, the third culture environment, and the fourth culture environment is serum-free and/or animal component free.

In one embodiment, one or more of the second culture medium, the third culture medium, and the fourth culture medium lacks an exogenously added inhibitor of BMP signaling.

In one embodiment, one or more the first culture medium, the second culture medium, the third culture medium, and the fourth culture medium lacks nicotinamide.

In one embodiment, the population of PSC are cultured under non-adherent conditions to produce three-dimensional retinal organoids. In one embodiment, the population of PSC are seeded into a microwell device.

In other aspects of this disclosure, methods of differentiating immature or mature RPE do not begin from PSC, but rather begin from any downstream cell population described herein using the appropriate culture environments and/or media, also as described herein.

In another aspect of this disclosure are provided systems or kits for differentiating immature or mature RPE from a population of PSC (or any downstream intermediate), the system(s) or kit(s) comprising one or more of: stage-appropriate media compositions (or basal media and supplements); extracellular matrix protein(s); and instructions.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows step-wise differentiation of immature RPE from pluripotent stem cells. Differentiation of two iPS lines (3A and M001) was performed as described herein, and representative images were taken at day 2, 7 and 14 (A). Scale bars represent 200 μm. Flow cytometry analysis of day 14 cells for PMEL17 marker expression among PSC-derived immature RPE cells (B).

FIG. 2 shows cell morphology during RPE maturation. Differentiation of two PSC lines (1C and H9) was performed as described herein, and representative images were taken at day 18, 21, 28, 42 and 49. Scale bars represent 200 μm.

FIG. 3 shows the characterization of matured RPE. Flow cytometry results of PMEL17 (91.4%±1.2%) marker expression among day 14 differentiated cells, and PMEL17 (96.6%±0.5%) and RPE65 (93%±1.0%) marker expression among day 49 differentiated cells, derived from 4 different PSC lines (A). Data represent the mean of 22-25 experiments±standard error of the mean. Bar graphs quantifying apical and basal secretion of growth factors from mature RPE derived from 4 different PSC lines. Day 52 cells were assessed for apical and basal secretion of VEGF (B) and day 53 cells were assessed for apical and basal secretion of PEDF (C). Bar graphs of barrier function of mature RPE differentiated from four different PSC lines, with transepithelial electrical resistance (TEER) values measured for day 49 cultures (D). For B)-D), data represent the mean of three experiments. Flow cytometry-plots quantifying phagocytosis of FITC-conjugated photoreceptor outer segments by matured RPE derived from two PSC lines (H9 and 1C) (E). Representative images of the pigmentation of matured RPE derived from four different PSC lines. Day 49 cells were imaged by bright field microscopy (F). Scale bars represent 50 μm.

FIG. 4 shows the effects of media composition during differentiation of immature RPE. Differentiation of two PSC lines (H9 and M001) was performed as described herein, but in the presence of 0.5×, 0.75×, 1× and 1.25× concentrations of cytokines and small molecules between days 4 and 14. Representative images were taken at day 14 (A) and day 49 cells (B). Bar graphs quantifying melanin content among day 49 cells in each medium composition is shown in (C). Day 14 and 49 cells were assessed for PMEL17 expression by flow cytometry (D). Data represent the mean of two experiments.

FIG. 5 shows the effect of different basal media on RPE maturation. Representative bright field images show the pigmentation of 1C-derived RPE cultured in two different supplemented and unsupplemented basal media formulations, compared to a control basal medium (A). Scale bars represent 200 μm. Bar graph of melanin content after culture in the supplemented and unsupplemented media formulation (B). Data represent the mean of 4 experiments±sem. Representative bright field images of H9-derived RPE cells cultured in the presence of increasing concentrations of ferric nitrate (10 μM, 25 UM and 50 μM) or ferric ammonium citrate (10 μM, 25 UM and 50 μM), compared to an unsupplemented medium control (C). Scale bars represent 200 μm. Bar graph of melanin content in the presence of the indicated concentrations of ferric nitrate or ferric ammonium citrate is shown (D).

FIG. 6 shows differentiation efficiencies of either single cell- or clump-passaged hPSCs. Differentiation efficiency of cells from 4 four different PSC lines was assessed by flow cytometry for PMEL17 marker expression. Data represent the mean of 8-18 experiments±sem.

FIG. 7 shows the efficiency of RPE differentiation using an alternative 3-stage protocol. Representative bright field images show cell morphology of 1C-derived RPE at day 14 and 49 (A). Flow cytometry analysis of MITF and RPE65 expression among day 49 WLS-1C- and H9-derived RPE (B). Flow cytometry analysis of PMEL17 expression among day 14 WLS-1C- and H9-derived immature RPE using the 3-stage protocol (C). Flow cytometry analysis of PMEL17 expression among day 14 WLS-1C- and H9-derived immature RPE cultured in the presence of a combination of alternate inhibitors of wnt signaling and of BMP signaling pathways (D).

FIG. 8 shows the efficiency of RPE differentiation using an alternative 5-stage protocol. Box and whisker plots summarize the frequency of immature RPE (day 14) derived from 1C and H9 cells, assessed by flow cytometry analysis of PMEL17 expression. Data represent the mean of 4-12 experiments±sem.

FIG. 9 shows box and whisker plots comparing the differentiation efficiencies of 5-stage, 3-satge and 4-stage RPE differentiation protocols. Data represent the mean of 6-31 experiments±sem.

FIG. 10 shows the effect of different BMP inhibitors on RPE differentiation efficiency. Percent positive immature RPE cells from 3A and H9 cell lines generated on day 14 were analysed by flow cytometry for PMEL17 marker expression (A). Immature RPE yield per well was calculated for 3A and H9 cell lines on day 14 using a hemocytometer (B).

FIG. 11 shows generation and characterization of retinal organoids. Brightfield images show organoid generation over time (A). Immunohistochemistry staining to characterize expression of various protein markers (SNCG, PAX6, VSX2, Prox1, AP2a, OTX2 and CRX) and a nuclear marker (DAPI) (B). Each denoted by arrows.

### DETAILED DESCRIPTION

This disclosure relates to media compositions and kits, and to methods for step-wise differentiation of retinal pigment epithelial cells (RPE) and/or progenitors thereof. More specifically, this disclosure relates to media compositions and kits, and to methods for step-wise differentiation of RPE-like cells and/or progenitors thereof beginning from one or more PSC.

Where used in the disclosure, the term “population of PSC” or “PSC” refers to one or more cells that are capable of self-renewal and also capable of differentiating to all three germ layers, i.e., ectoderm, mesoderm, and endoderm, and beyond. Conditions and media requirements for culturing PSC to maintain an undifferentiated state are known. PSC is a broad term that captures both embryonic stem cells (“ESCs”) and induced pluripotent stem cells (“iPSCs”), and the like. While there may be ethical challenges with sourcing ESCs, by now several ESC lines are well established in the field. On the other hand, iPSCs may be induced from a great variety of readily accessible cells, including adult somatic cells, and are therefore associated with fewer ethical constraints. Thus, new iPSC lines emerge with regularity. PSCs are an important model to study differentiation mechanisms, to model diseases, and offer significant medical opportunities. PSCs may be obtained, derived or induced from any source species, but in this disclosure, PSCs are preferably human.

Where used in this disclosure, the term “population of cells fated for the retinal lineage” refers to a group of cells differentiated from one or more precursors (e.g. PSC), but still retain various potentials (albeit more limited relative to the one or more precursors). A population of cells fated for the retinal lineage indicates a population of ectoderm or mesoderm cells that have the ability to differentiate further into RPE cells: cells fated for retinal pigmented epithelial cell fate; RPE precursor cells; or cells differentiating towards the eye field via an anterior neural ectodermal state. In the context of this disclosure, a population of cells fated for the retinal lineage may be biased toward the ectodermal lineage or the mesodermal lineage or may have acquired ectodermal potential or mesodermal potential. One or more subsequent differentiation steps are required to differentiate a population of cells fated for the retinal lineage to immature/mature retinal (epithelial) cells. Characteristics of a cell fated for the retinal lineage include, but are not limited to, a loss of high OCT4, TRA-1-60, and/or NANOG expression. Further, a cell fated for the retinal lineage may begin to acquire expression of relevant markers, such as with respect to SIX3, SIX6, LHX2, PAX6, RAX, or NR2E1 levels. In one embodiment, cells fated for the retinal lineage efficiently generate or differentiate to immature and/or mature RPE cells.

Where used in this disclosure, the term “population of eye field progenitors” refers to a group of cells differentiated from one or more cells fated for the retinal lineage, but still retain various potentials. A population of eye field progenitors may be biased toward the ectodermal lineage or may have acquired ectodermal potential. Also, a population of eye field progenitors may comprise eye-field specialized cells, eye-field progenitor cells, anterior neural plate-derived cells or RPE precursor cells. One or more subsequent differentiation steps are required to differentiate a population of eye field progenitors to immature/mature RPE. The population of eye field progenitors may express eye-field transcription factors such as PAX6, RAX, SIX3 and LHX2. While a population of cells fated for the retinal lineage may have some overlap with a population of eye field progenitors, the latter may encompass a consolidation or homogenization of relevant marker expression, such as with respect to SIX3, SIX6, LHX2, PAX6, RAX, or NR2E1 levels. Further, eye field progenitors may begin to express PMEL17, such as at relatively low levels (in comparison to immature RPE). In one embodiment, eye field progenitors efficiently generate or differentiate to immature and/or mature RPE cells.

Where used in this disclosure, the term “population of optic vessel progenitors” refers to a group of cells differentiated from a developmentally earlier precursor, such as one or more eye field progenitors. Optic vessel progenitors may possess a more limited potential relative to eye field progenitors, and may develop or differentiate into the optic vesicle and the optic cup. Optic vesicles may comprise retinal stem cells (e.g. cells capable of generating all the neuroectoderm-derived cells of the eye) and/or transient bipotent progenitors (e.g. cells that give rise to retinal pigment epithelium or neural retina). A population of optic vessel progenitors are biased toward the ectodermal lineage and have acquired ectodermal potential. A population of optic vessel progenitors may comprise optic vessel specialized cells, optic vessel progenitor cells, cells interacting with surface ectoderm or RPE lineage committed cells, or RPE precursor cells. One or more subsequent differentiation steps may be required to differentiate a population of optic vessel progenitors to immature/mature RPE. While a population of eye field progenitors may have some overlap with a population of optic vessel progenitors, the latter may exhibit (relative to a population of eye field progenitors) a consolidation or homogenization of relevant marker expression, such as with respect to MITF or VSX2 levels. Further, optic vessel progenitors may express higher levels of PMEL17 and/or PAX6 than eye field progenitors. In one embodiment, optic vessel progenitors efficiently generate or differentiate to immature and/or mature RPE cells.

Where used in this disclosure, the term “population of immature retinal pigment epithelial cells” or “immature RPE” refers to a group of cells differentiated from a developmentally earlier precursor, such as one or more optic vessel progenitors. Immature RPE possess a more limited potential relative to optic vessel progenitors. A population of immature RPE may be biased toward, or may be limited to, the RPE lineage. A population of immature retinal pigment epithelial cells may comprise cells fated to become mature RPE or intermediate RPE progenitor stage cells. A population of immature RPE may be derived or differentiated from RPE precursor cells. One or more subsequent differentiation/maturation steps may be required to differentiate immature RPE to mature RPE. While a population of immature RPE may have some overlap with a population of optic vessel progenitors, the former may encompass a consolidation or homogenization of relevant marker expression, such as with respect to PMEL17, MITF or PAX6 levels. Immature RPE may be characterized by a loss of RAX and/or CHX10 expression, and an emergence of one or more of RPE65, ZO1, TYR, and TYRP1 expression. Immature RPE, and in particular PSC-derived immature RPE, may exhibit at least some of the hallmarks of mature (d) RPE, such as polygonal morphology and pigmentation. In one embodiment, immature RPE efficiently generate or differentiate to mature RPE.

Where used in this disclosure, the term “population of mature (d) retinal pigment epithelial cells” or “mature (d) RPE” refers to a group of cells differentiated from a developmentally earlier precursor, such as one or more immature RPE. A population of immature RPE may be derived or differentiated from RPE precursor cells. When derived from one or more PSC, RPE may or may not fully mature, but nevertheless exhibit many characteristics of primary mature RPE, thus when used herein the term mature RPE may similarly refer to matured or maturing RPE. Mature (d) RPE possess a more limited potential relative to immature RPE and may be terminally differentiated. A population of mature (d) RPE are biased toward, and may be limited to, the RPE lineage. While a population of mature (d) RPE may have some overlap with a population of immature RPE, the former may encompass a consolidation or homogenization of relevant marker expression, such as with respect to RPE65, TYR, TYRP1, BEST1, CRALBP, EZRIN, and ZO-1 levels. Further, mature (d) RPE, and in particular PSC-derived mature (d) RPE, may exhibit at least some or all of the hallmarks of mature RPE, such as polygonal morphology, pigmentation, ability to phagocytose photoreceptor outer segments, polarity, and post-mitotic.

Where used in this disclosure, the term “RPE precursor cells” refers to any of a population of cells fated for the retinal lineage, a population of eye field progenitors and/or a population of optic vessel progenitors.

Where used in this disclosure, the term “neural retina” refers to a heterogenous group of cells or structure that are bordered by the RPE and which can comprise cell types including retinal progenitor cells, retinal ganglion cells, photoreceptor progenitor cells, rod and cone photoreceptor cells, bipolar cells, amacrine cells, retinal ganglion cells, muller glia cells and horizontal cells

### Methods

In one aspect of this disclosure are provided methods of differentiating a population of cells fated for the retinal lineage. In one aspect of this disclosure are provided methods of differentiating a population of cells fated for the retinal lineage, and beyond to immature and/or mature RPE. In one embodiment, differentiating immature and/or mature RPE proceeds through more than one intermediate cell population, as further described below.

Methods of this disclosure may involve providing a population of PSC and exposing such population to a first culture environment. Since various PSC lines have been published and/or made commercially available, and the technology for generating PSC lines is by now routine, the population of PSC is not particularly limited. Preferably, the population of PSC are undifferentiated or substantially undifferentiated, that is the population of PSC possess the ability to differentiate to all germ layers. In one embodiment, the population of PSC are iPSC or ESC.

The population of PSC may be wild type, or may be mutated or edited at one or more genomic loci. In some embodiments, it may be desirable to model diseases of the retina or other neurological disease, and thus PSC harboring one or more mutation(s) of interest (present in the reprogrammed cell(s) or accordingly edited) would be an appropriate start point.

The population of PSC may be derived from any animal or mammalian species. In a preferred embodiment, the population of PSC are derived from a human, primate, or rodent source. To avoid or limit variability among downstream differentiated cells, such as immature or mature (d) RPE, it may be desirable that the population of PSC are clonally derived. The population of PSC may be derived from a human patient or may be a patient-derived iPSC line.

As indicated above, methods of this disclosure comprise exposing a population of PSC to a first culture environment. The first culture environment is not particularly limited provided it is either supportive of the population of PSC, or supportive of differentiating the population of PSC to a downstream lineage (toward the retinal lineage). In one embodiment, the first culture environment comprises one or both of an extracellular matrix protein and an inhibitor of BMP signaling. In one embodiment, the first culture environment comprises one or both of an extracellular matrix protein and a first culture medium, wherein an inhibitor of BMP signaling is comprised in the first culture medium.

Extracellular matrix proteins for culturing/supporting PSC are known, and commercially available. An extracellular matrix protein used in a first culture environment of this disclosure is not limited provided, that it supports the population of PSC and also that it does not inhibit the differentiation of PSC to a downstream population of interest, such as a population of cells fated for the retinal lineage.

The extracellular matrix protein may be coated on a culture surface (of a first culture environment). The culture surface may be any surface for culturing PSC and to differentiate PSC to a downstream ectodermal (e.g. neural and/or retinal) lineage. By way of non-limiting example, the surface may be a wall (e.g., a bottom wall) of a culture flask, cell culture vessel, petri dish, roller bottle, cell culture dish, multi-well plate, or microcarriers. In one embodiment, the cell culture dish may be untreated and have a hydrophobic surface or may be treated to have a hydrophilic and negatively charged surface. Further, the surface may be a membrane, filter, or any other type of porous surface that supports a population of cells seeded thereon, such as a cell culture insert (e.g., Transwell™ insert, or the like).

In one embodiment, the one or more extracellular matrix (ECM) proteins are coated on the culture surface prior to seeding the population of PSC. In one embodiment, the one or more ECM proteins may be comprised in a cell culture medium that is applied to the culture surface. In such an embodiment, the one or more ECM proteins may become coated on the surface as the population of PSC settle.

In one embodiment, one or more ECM proteins are added directly to cell culture medium. In one embodiment, a desired quantity or concentration of one or more ECM proteins may be added to a cell culture medium comprising a population or suspension of cells. In one embodiment, a desired quantity or concentration of one or more ECM proteins may be added to a cell culture medium that later comes into contact with a population or suspension of cells.

By way of non-limiting example, the one or more extracellular matrix protein may be, or is selected from, a collagen, a laminin, a decorin, a vitronectin, a fibronectin, Synthemax™, Synthemax™ Ila, Matrigel®, or a mixture comprising more than one of the foregoing. In one embodiment, the extracellular matrix protein is one of vitronectin, laminin, fibronectin, or collagen. In one embodiment, the extracellular matrix protein is any combination or mixture of vitronectin, laminin, fibronectin, or collagen.

In embodiments comprising a collagen, the collagen may be one or more of type I collagen, type II collagen, type Ill collagen or type IV collagen. In embodiments comprising a laminin, the laminin may be one or more of laminin-111, laminin-211, laminin-121, laminin-221, laminin-332, laminin-311, laminin-321, laminin-411, laminin-421, laminin-511, a fragment of laminin-511 (E.g., laminin 511-E8), laminin-521 or laminin-213. In embodiments comprising an elastin, the elastin may be one or more of elastin or tropoelastin. In embodiments comprising a nidogen (also known as entactin), the nidogen may be one or more of nidogen-1 or nidogen-2. In one embodiment, the extracellular matrix protein may be vitronectin, or an isoform thereof. In one embodiment, the extracellular matrix protein may be Matrigel®.

In one embodiment, ECM proteins may be recombinant and/or of natural origin. In one embodiment, ECM proteins may be genetically engineered (e.g. fusion proteins). In one embodiment, ECM proteins may be a whole protein or a fragment thereof, such as a peptide fragment.

Methods of this disclosure may involve ECM protein(s) coated on a culture surface or added directly to a cell culture medium. Concentrations of ECM protein(s) are not particularly limited. In one embodiment, a concentration of an extracellular matrix protein may range between about 0.1 μg/mL to 1 mg/mL. In one embodiment, the concentration of the one or more (or each) ECM proteins ranges between about 1 ng/ml to 1 μg/mL, about 100 ng/ml to 100 μg/mL, about 500 ng/ml to 50 μg/mL, or about 1 μg/mL to 30 μg/mL. In embodiments where the ECM protein is a combination of two or more pre-mixed ECM proteins, such as Matrigel™, may be diluted about 1:10, 1:20, 1:30, 1:40, 1:50, 1:60, 1:70, 1:80, 1:90, 1:100, or more.

As indicated herein, a first culture environment may comprise an inhibitor of BMP signaling. By way of non-limiting example, the inhibitor of BMP signaling may be a small molecule, a peptide or a protein. Examples of small molecule inhibitors of BMP signaling include, but are not limited to: 4-[6-[4-(1-piperazinyl)phenyl]pyrazolo[1,5-a]pyrimidin-3-yl]-quinoline dihydrochloride (LDN-193189), 6-[4-[2-(1-piperidinyl) ethoxy]phenyl]-3-(4-pyridinyl)-pyrazolo[1,5-a]pyrimidine (dorsomorphin), 4-[6-(4-propan-2-yloxyphenyl) pyrazolo[1,5-a]pyrimidin-3-yl]quinoline (DMH1), 4-[6-[4-[2-(4-Morpholinyl) ethoxy]phenyl]pyrazolo[1,5-a]pyrimidin-3-yl]-quinoline (DMH2), 3-[(6-Amino-5-(3,4,5-trimethoxyphenyl)-3-pyridinyl]phenol (K02288) and 5-[6-(4-methoxyphenyl) pyrazolo[1,5-a]pyrimidin-3-yl]-quinoline (ML-347). Examples of protein inhibitors of BMP signaling include, but are not limited to: noggin, chordin, gremlin, crossveinless-2 (CV2), USAG-1 (uterine sensitization-associated gene-1), follistatin and sclerostin (also known as SOST).

A concentration of the inhibitor of BMP signaling in the first culture environment may influence the efficiency of differentiating a population of PSC, through one or more intermediates, to immature or mature (d) RPE. In one embodiment, the concentration of the inhibitor of BMP signaling in the first culture environment is critical for most efficiently influencing differentiation of the population of PSC, through one or more intermediates, to immature or mature (d) RPE. In one embodiment, the concentration of the inhibitor of BMP signaling ranges between about 0.01 nM to 100 μM, about 0.05 nM to 75 μM, about 0.1 nM to 50 μM, about 0.15 nM to 25 μM, about 0.2 nM to 10 μM, about 0.25 nM to 5 μM. In one embodiment, the concentration of the inhibitor of BMP signaling is below 25 μM, below 10 μM, below 5 μM, below 2.5 μM, below 2 μM, below 1 μM, below 0.75 μM, below 0.5 μM, below 0.25 μM, below 0.1 μM, below 0.075 μM, below 0.05 μM, or below 0.025 μM. In one embodiment, the concentration of the inhibitor of BMP signaling ranges between about 20 nM to 750 nM. In one embodiment, the concentration of the inhibitor of BMP signaling ranges between about 5 ng/ml to 150 ng/ml.

In certain embodiments, the first culture environment (e.g., the first culture medium) is free of one or both of i) a wnt signaling antagonist and ii) an agonist of activin signaling. In one embodiment, the first culture environment may be free of a wnt signaling antagonist but contain an agonist of activin signaling. In one embodiment, the first culture environment may be free of an agonist of activin signaling but contain a wnt signaling antagonist. In one embodiment, the first culture environment does not contain either a wnt signaling antagonist and an agonist of activin signaling. In one embodiment, the first culture environment may contain both a wnt signaling antagonist as well as an agonist of activin signaling.

By way of non-limiting example, a wnt signaling antagonist may be a small molecule, a cytokine, a peptide or a protein. Examples of small molecule wnt signaling antagonists include, but are not limited to, N-(6-chloro-1,3-benzothiazol-2-yl)-3-(3,4-dimethoxyphenyl) propenamide (KYO2111), 2-[4-(trifluoromethyl)phenyl]-1,5,7,8-tetrahydrothiopyrano[4,3-d]pyrimidin-4-one (XAV939), 4-[(3aR,4S,7R,7aS)-1,3,3a,4,7,7a-hexahydro-1,3-dioxo-4,7-methano-2H-isoindol-2-yl]-N-8-quinolinylbenzamide (IWR-1-endo), N-(6-methyl-2-benzothiazolyl)-2-[[3,4,6,7-tetrahydro-3-(2-methoxyphenyl)-4-oxothieno[3,2-d]pyrimidin-2-yl]thio]-acetamide (IWP-4), 2-[[3-(4-fluorophenyl)-3,4,6,7-tetrahydro-4-oxothieno[3,2-d]pyrimidin-2-yl]thio]-N-(6-methyl-2-benzothiazolyl)-acetamide (IWP-3) and N-(6-methyl-2-benzothiazolyl)-2-[(3,4,6,7-tetrahydro-4-oxo-3-phenylthieno[3,2-d]pyrimidin-2-yl)thio]-acetamide (IWP-2). Examples of a cytokine or protein-based wnt signaling antagonist include, but are not limited to, Insulin-like growth factor-binding protein 4 (IGFBP-4), Dickkopf-related protein 1 (DKK-1), DKK-2, DKK-3, DKK-4, Soggy-1/DkkL1, Secreted Frizzled Related Proteins (sFRP), APCDD1, APCDD1L, Draxin, LMBR1L, Notum, SOST/Sclerostin, USAG1 and WIF-1.

By way of non-limiting example, an agonist of activin signaling may be a small molecule, a cytokine, a peptide or a protein. Examples of an agonist of activin signaling include, but are not limited to, Activin A, 1-(2-cyclopentylidenehydrazide)-heptanedioic acid (IDE2) and 1-[2-[(2-carboxyphenyl)methylene]hydrazide]-heptanedioic acid (IDE1).

A population of PSC may be exposed to (e.g. cultured in) the first culture environment for between about 0 and 120 hours. The duration of exposure to the first culture environment (and the inhibitor of BMP signaling) may be as important or critical to deriving a population of cells fated for the retinal lineage (or a downstream population such as eye field progenitors, optic vessel progenitors, and/or immature RPE), as is the concentration of the inhibitor of BMP signaling. In one embodiment, the population of PSC are cultured in the first culture environment for about 96 hours or less. In one embodiment, the population of PSC are cultured in the first environment for about 72 hours or less. In one embodiment, the population of PSC are cultured in the first environment for about 60 hours or less. In one embodiment, the population of PSC are cultured in the first environment for between about 0 to 60 hours. In one embodiment, the population of PSC are cultured in the first environment for about 48 hours, or about 48 hours±5 hours.

A first culture medium forming a part of the first culture environment may comprise a basal medium. Basal media typically include one or more of: amino acids, vitamin(s), organic and/or inorganic salt(s), buffer(s), antioxidant(s), energy (e.g., carbon) source(s), and the like for supporting the growth of cells. In some embodiments, basal media do not include one or more of the foregoing components, and if essential may be supplemented. Numerous commercially available basal media are known, including Dulbecco's Modified Eagle's Medium (DMEM), F12, Roswell Park Memorial Institute Medium (RPMI) 1640, Iscove's Modified Dulbecco's Medium (IMDM), Advanced DMEM, Advanced DMEM/F-12, Immunocult™-branded media, STEMSpan™-branded media, X-VIVO-branded media. In one embodiment, the basal medium is an Immunocult™-branded medium or a STEMSpan™-branded medium.

After culturing the population of PSC in the first culture environment under conditions and for a duration as described herein, a population of cells fated for the retinal lineage may be derived (e.g. cells that are no longer PSC and are along the path toward ectoderm or already are ectodermal).

Methods of this disclosure may further comprise exposing the population of cells arising from the first culture environment (e.g. a population of stage 1 cells, or a population of cells fated for the retinal lineage) to a second culture environment. Exposure of such cells to a second culture environment may cause them to further differentiate to or further along the path toward immature and/or mature (d) RPE. In one embodiment, a downstream population of cells derived from the population arising from the first culture environment comprise a population of eye field progenitors.

A second culture environment may comprise an extracellular matrix protein, as described above, whether the same as or different from the first culture environment. In one embodiment, a second culture environment comprises a second cell culture medium. In one embodiment, a second culture environment comprises both an extracellular matrix (as described above) and a second culture medium.

A second cell culture medium may also comprise a basal medium, as described above. In one embodiment, the basal medium is an Immunocult™-branded or a STEMSpan™-branded medium. In one embodiment, the basal medium is an X-VIVO-branded medium. In one embodiment, the basal medium is an equivalent of or alternative to an Immunocult™-, or a STEMSpan™-, or an X-VIVO-branded medium.

A second cell culture medium, and a basal medium thereof, will be appropriately supplemented, such as to differentiate a population of cells (fated for the retinal lineage) to or toward immature and/or mature (d) RPE. In one embodiment, a second cell culture medium comprises an insulin-like growth factor (e.g. IGF-1 or IGF-2). In one embodiment, a second cell culture medium comprises an agonist of FGF signaling, such as a member of the FGF family. In one embodiment, an agonist of FGF signaling is basic fibroblast growth factor (FGFb). In one embodiment, a second cell culture medium comprises a basal medium, an IGF, and an agonist of FGF signaling.

In one embodiment, the second culture environment and/or the second culture medium comprises an inhibitor of BMP signaling, of the types and concentrations as described above. In one embodiment, the second culture environment and/or the second culture medium is free of an inhibitor of BMP signaling. In one embodiment, the second culture environment and/or the second culture medium is free of an exogenously added inhibitor of BMP signaling.

A population of cells (e.g. fated for the retinal lineage) exposed to the second culture environment may be cultured therein for between about 0 and 120 hours. The duration of exposure to the second culture environment (and the presence of the inhibitor of BMP signaling) may be as important or critical to deriving a population of cells fated for the retinal lineage (or a downstream population such as eye field progenitors, optic vessel progenitors, and/or immature RPE), as is the concentration of factors in the second culture medium. The duration of exposure to the second culture environment (and the absence of the inhibitor of BMP signaling) may be as important or critical to deriving a population of cells fated for the retinal lineage (or a downstream population such as eye field progenitors, optic vessel progenitors, and/or immature RPE), as the absence of the inhibitor of BMP signaling.

In one embodiment, the population of cells (fated for the retinal lineage) are cultured in the second culture environment for about 96 hours or less. In one embodiment, the population of cells (fated for the retinal lineage) are cultured in the second culture environment for about 72 hours or less. In one embodiment, the population of cells (fated for the retinal lineage) are cultured in the second culture environment for about 60 hours or less. In one embodiment, the population of cells (fated for the retinal lineage) are cultured in the second culture environment for between about 0 to 60 hours. In one embodiment, the population of cells (fated for the retinal lineage) are cultured in the second culture environment for between about 12 to 60 hours. In one embodiment, the population of cells (fated for the retinal lineage) are cultured in the second culture environment for about 48 hours, or about 48 hours±5 hours.

After culturing the arising population of cells (fated for the retinal lineage) in the second culture environment under conditions and for a duration as described herein, a population of progenitors (e.g. eye field progenitors, or a population of stage 2 cells, or a second population of differentiated cells) may be derived. Such population may comprise all the progenitors of the neural-derived eye structures. Exemplary markers of such population include, but are not limited to, PAX6, RAX, SIX3, SIX6 (Optx2), LHX2, NR2E1, ET and tll.

Methods of this disclosure may further comprise exposing the population of cells arising from the second culture environment (e.g. a population of stage 2 cells, or a population of eye field progenitors) to a third culture environment. Exposure of such cells to a third culture environment may cause them to still further differentiate to or further along the path toward immature and/or mature (d) RPE. In one embodiment, a downstream population of cells derived from the population arising from the third culture environment comprise a population of optic vessel progenitors (e.g. a population of stage 3 cells, or a third population of cells).

A third culture environment may comprise an extracellular matrix protein, as described above, whether the same as or different from the first and/or second culture environment. In one embodiment, a third culture environment comprises a third cell culture medium. In one embodiment, a third culture environment comprises both an extracellular matrix (as described above) and a third culture medium.

A third cell culture medium may also comprise a basal medium, as described above. In one embodiment, the basal medium is an Immunocult™-branded or a STEMSpan™-branded medium. In one embodiment, the basal medium is an X-VIVO-branded medium. In one embodiment, the basal medium is an equivalent of or alternative to an Immunocult™-, or a STEMSpan™-, or an X-VIVO-branded medium.

A third cell culture medium, and a basal medium thereof, will be appropriately supplemented in order to differentiate a population of stage 2 cells (e.g. a population of eye field progenitors) to or toward immature and/or mature (d) RPE. In one embodiment, a third cell culture medium comprises an insulin-like growth factor (e.g., IGF-1 or IGF-2). In one embodiment, a third cell culture medium comprises an agonist of activin signaling, as described above. In one embodiment, a third cell culture medium comprises a basal medium, an IGF, and an agonist of FGF signaling.

A population of cells (e.g eye field progenitors) exposed to a third culture environment may be cultured in the third culture environment for between about 1 and 10 days. In one embodiment, the (eye field) progenitors are cultured in the third culture environment for between about 1 and 7 days. In one embodiment, the (eye field) progenitors are cultured in the third culture environment for between about 1 and 5 days. In one embodiment, the (eye field) progenitors are cultured in the third culture environment for between about 2 and 5 days. In one embodiment, the (eye field) progenitors are cultured in the third culture environment for between about 2 and 4 days. In one embodiment, the (eye field) progenitors are cultured in the third culture environment for about 3 days, or about 4 days.

After culturing the population of (eye field) progenitors in the third culture environment under conditions and for a duration as described herein, a population of progenitors (e.g. a optic vessel progenitors, a population of stage 3 cells, or a third population of differentiated cells) may be derived. Such population may comprise cells that are bipotent, such as for retinal pigment epithelium and/or neural retina. Such population of cells may further develop or differentiate into an optic vesicle and/or an optic cup. Such population of cells may be characterized by expression of lineage-specific markers, such as MITF and OTX2.

Methods of this disclosure may further comprise exposing the population of cells arising from the third culture environment (e.g. a population of stage 3 cells, or a population of optic vessel progenitors) to a fourth culture environment. Exposure of such cells to a fourth culture environment may cause them to still further differentiate to or further along the path toward immature and/or mature (d) RPE. In one embodiment, a downstream population of cells derived from the population arising from the fourth culture environment comprise a population of immature RPE.

In one embodiment, a fourth culture environment comprises an extracellular matrix protein, as described above, whether the same as or different from the first and/or second culture environment. In one embodiment, a fourth culture environment comprises a fourth cell culture medium. In one embodiment, a fourth culture environment comprises both an extracellular matrix (as described above) and a fourth culture medium.

A fourth cell culture medium may also comprise a basal medium, as described above. In one embodiment, the basal medium is an Immunocult™-branded or a STEMSpan™-branded medium. In one embodiment, the basal medium is an X-VIVO-branded medium. In one embodiment, the basal medium is an equivalent of or alternative to an Immunocult™-, or a STEMSpan™-, or an X-VIVO-branded medium.

A fourth cell culture medium, and a basal medium thereof, will be appropriately supplemented in order to differentiate a population of arising (optic vessel) progenitors to or toward immature and/or mature (d) RPE. In one embodiment, a fourth cell culture medium comprises one or more of an agonist of activin signaling, as described above, an agonist of wnt signaling, and an antagonist of FGF signaling. In one embodiment, a fourth cell culture medium may further comprise a RHO/ROCK pathway inhibitor. Such a RHO/ROCK pathway inhibitor may be any compound or molecule that inhibits the rho kinase or ROCK pathway.

By way of non-limiting example, an agonist of wnt signaling may be a small molecule, a cytokine, a peptide or a protein. Examples of small molecule agonists of wnt signaling include, but are not limited to, 3-[3-[(acetyloxy)imino]-1,3-dihydro-2H-indol-2-ylidene]-6-bromo-1,3-dihydro-2H-indol-2-one (BIO-Acetoxime), 9-bromo-7,12-dihydro-indolo[3,2-d][1]benzazepin-6 (5H)-one (Kenpaullone), 3-[[6-(3-aminophenyl)-7H-pyrrolo[2,3-d]pyrimidin-4-yl]oxy]phenol (TWS119), N6-[2-[[4-(2,4-dichlorophenyl)-5-(1H-imidazol-2-yl)-2-pyrimidinyl]amino]ethyl]-3-nitro-2,6-pyridinediamine (CHIR98014), 3-(2,4-Dichlorophenyl)-4-(1-methyl-1H-indol-3-yl)-1H-pyrrole-2,5-dione (SB216763), 2-[2-(4-acetylphenyl)diazenyl]-2-(3,4-dihydro-3,3-dimethyl-1 (2H)-isoquinolinylidene)-acetamide (IQ-1), 6-[[2-[[4-(2,4-dichlorophenyl)-5-(5-methyl-1H-imidazol-2-yl)-2-pyrimidinyl]amino]ethyl]amino]-3-pyridinecarbonitrile (CHIR99021) and 6-bromo-3-[(3E)-1,3-dihydro-3-(hydroxyimino)-2H-indol-2-ylidene]-1,3-dihydro-(3Z)-2H-indol-2-one (BIO).

By way of non-limiting example, an antagonist of FGF signaling may be a small molecule, a cytokine, a peptide or a protein. Examples of antagonists of FGF signaling include, but are not limited to, 2-[(1,2-dihydro-2-oxo-3H-indol-3-ylidene)methyl]-4-methyl-1H-pyrrole-3-propanoic acid (SU5402) and N-[2-[[4-(diethylamino)butyl]amino]-6-(3,5-dimethoxyphenyl)pyrido[2,3-d]pyrimidin-7-yl]-N′-(1,1-dimethylethyl)-urea (PD173074).

A population of optic vessel progenitors (e.g. a population of stage 3 cells) exposed to the fourth culture environment may be cultured in the fourth culture environment for between about 3 and 12 days. In one embodiment, the arising progenitors are cultured in the fourth culture environment for between about 3 and 10 days. In one embodiment, the arising progenitors are cultured in the fourth culture environment for between about 5 and 10 days. In one embodiment, the arising progenitors are cultured in the fourth culture environment for between about 6 and 9 days. In one embodiment, the arising progenitors are cultured in the fourth culture environment for between about 6 and 8 days. In one embodiment, the arising progenitors are cultured in the fourth culture environment for about 6 days, 7 days, or 8 days.

The concentration of cytokines and small molecules present in the third and fourth culture media may be in the range of 2-150 ng/ml or 1-15 M. In one embodiment, the concentration of cytokine(s) in the third and fourth media may be 50 ng/ml, 75 ng/ml, 100 ng/ml or 125 ng/ml. In one embodiment, the concentration of cytokine(s) in the third media may be 5 ng/ml, 7.5 ng/ml, 10 ng/ml or 12.5 ng/ml. In one embodiment, the concentration of small molecule(s) in the fourth media may be 5 μM, 7.5 μM, 10 UM or 12.5 μM. In one embodiment, the concentration of small molecule(s) in the fourth media may be 1.5, 2.25, 3 or 3.75 μM. In one embodiment, the cytokine(s) present in the third medium may be Activin A or IGF-1. In one embodiment, the cytokine(s) present in the fourth medium may be Activin A.

After culturing the population of (optic vessel) progenitors in the fourth culture environment under conditions and for a duration as described herein, a population of immature RPE (e.g. a population of stage 4 cells, or a fourth population of differentiated cells) may be derived. A population of immature RPE may be fated to become RPE cells. In one embodiment, immature RPE comprise cells that have started differentiating to or toward mature RPE cells but have not yet acquired all the characteristics of mature RPE cells. In one embodiment, immature RPE may exhibit certain characteristics such as cell surface marker expression and morphology similar to mature RPE cells.

In one aspect, or in an embodiment of the foregoing, methods of this disclosure may comprise dissociating a population of immature RPE. In one embodiment, methods of this disclosure comprise dissociating the population of immature RPE and replating the dissociated cells.

Immature RPE dissociated from a substrate may be enriched (such as by manual dissection, particle-based enrichment, or ligand-mediated enrichment) prior to replating the dissociated cells. In one embodiment, the simple act of dissociating a population of immature RPE and replating the dissociated cells enriches for immature RPE. Thus, in one embodiment, the dissociated population of immature RPE are not enriched other than by re-plating the bulk population of dissociated immature RPE (or a fraction thereof depending on the surface area of the culture surface into which they are replated). As such a dissociated population of RPE need not be enriched by manual dissection, particle-based enrichment, or ligand-mediated enrichment. Without being bound by theory, immature RPE may become enriched following dissociation and replating, by virtue of a reduced ability of contaminating cells to detach from a substrate or to reattach to a new substrate, or may be outcompeted by immature RPE for the surface area of a culture surface into which they are replated.

In one embodiment, an absence of serum in a (e.g., enrichment/replating) medium reduces or limits the establishment of contaminating cell types (such as neural retinal cells and/or corneal cells) in comparison to a serum-containing condition into which dissociated RPE are replated.

In one embodiment, a replating medium (e.g. an enrichment and/or maturation medium) comprises a RHO/ROCK pathway inhibitor, and such medium helps to (preferentially) promote the survival/attachment of PMEL17+ immature RPE in comparison to contaminating cells types. In one embodiment, a RHO/ROCK pathway inhibitor may be added as a supplement to an enrichment/replating/maturation medium. In one embodiment, dissociated immature RPE cells are cultured in an enrichment/replating/maturation medium comprising a RHO/ROCK pathway inhibitor for between 2 to 10 days.

Populations of immature RPE differentiated (and optionally enriched) as described herein may express characteristic markers. In one embodiment, about 50% or more of the population of immature RPEs express premelanosome protein (PMEL17). In one embodiment, 60% or more of the population of immature RPE express PMEL17. In one embodiment, 70% or more of the population of immature RPE express PMEL17. In one embodiment, 80% or more of the population of immature RPE express PMEL17. In one embodiment, 90% or more of the population of immature RPE express PMEL17. In one embodiment, 90-95% or more of the population of immature RPE express PMEL17.

In another aspect, or in an embodiment of the foregoing, methods of this disclosure may comprise maturing a population of immature RPE through contact with a maturation medium. Maturation media comprise at least a soluble source of iron. Examples of a soluble source of iron may include, but are not limited to, ferric nitrate and ferric ammonium citrate. In one embodiment.

In one embodiment, maturation media comprise a basal medium, as described above. In one embodiment, the basal medium is an Immunocult™-branded or a STEMSpan™-branded medium. In one embodiment, the basal medium is an X-VIVO-branded medium. In one embodiment, the basal medium is an equivalent of or alternative to an Immunocult™-, or a STEMSpan™-, or an X-VIVO-branded medium. Other examples of basal medium are known and may be substituted for the foregoing basal media.

Maturation media may further comprise one or more of a steroid hormone and a polyamine. In one embodiment, maturation media may further comprise progesterone and putrescine. In one embodiment, maturation media are xeno-free or animal-component-free. In one embodiment, maturation media does not comprise a RHO/ROCK pathway inhibitor.

A population of immature RPE exposed to a soluble source of iron, such as through contact with a maturation medium of this disclosure, may be cultured/matured for between about 1 to 10 weeks, or between about 1 to 7 weeks, or between about 1 to 5 weeks.

Following culture/maturation of a population of immature RPE in the presence of a soluble source of iron, the mature/matured/maturing RPE may begin expressing or become more uniform for markers of mature RPE. In one embodiment, a marker of mature RPE is RPE65. In one embodiment, 50% or more of the mature or matured or maturing RPE express RPE65. In one embodiment, 60% or more of the mature or matured or maturing RPE express RPE65. In one embodiment, 70% or more of the mature or matured or maturing RPE express RPE65. In one embodiment, 80% or more of the mature or matured or maturing RPE express RPE65. In one embodiment, 90% or more of the mature or matured or maturing RPE express RPE65. In one embodiment, 90-95% or more of the mature or matured or maturing RPE express RPE65.

Methods of this disclosure may be performed under animal component-free or serum-free conditions. In one embodiment, methods of this disclosure are performed under animal component-containing or serum-containing conditions. In one embodiment, the first culture environment, the second culture environment, the third culture environment, and the fourth culture environment is serum-free. In one embodiment, the first culture environment, the second culture environment, the third culture environment, and the fourth culture environment is serum-containing. In one embodiment, the first culture environment, the second culture environment, the third culture environment, and the fourth culture environment is animal component-containing. In one embodiment, the first culture environment, the second culture environment, the third culture environment, and the fourth culture environment is animal component-free.

Methods of this disclosure may be performed under feeder-free conditions, or in the absence of feeder cells. In one embodiment, the first culture environment, the second culture environment, the third culture environment, and the fourth culture environment is feeder-free or does not contain any feeder cells. In one embodiment, the population of PSC may be cultured under feeder-free conditions or in the absence of any feeder cells. In one embodiment, the population of PSC may be differentiated under feeder-free conditions or in the absence of any feeder cells.

In one embodiment, one or more of a second culture medium, a third culture medium, and a fourth culture medium lacks an exogenously added inhibitor of BMP signaling.

In one embodiment, one or more of a first culture medium, a second culture medium, a third culture medium, and a fourth culture medium lacks nicotinamide. In one embodiment, the second culture medium, the third culture medium, and the fourth culture medium lacks nicotinamide. In one embodiment, the third culture medium and/or the fourth culture medium lacks nicotinamide.

In some aspects, the disclosure relates to the use of the RPE cells derived by the methods of this disclosure for treating retina-associated diseases. Such retina-associated diseases may comprise retinal diseases or disorders or conditions related to retinal dysfunction, retinal injury or loss of RPE function. A non-limiting list of conditions that may be treated with the RPE cells of the invention comprises age-related macular degeneration (AMD), glaucoma, cataracts, retinitis pigmentosa (RP), Stargardt disease (SD), Proliferative vitreoretinopathy (PVR), Diabetic retinopathy (DR), lebers congenital amaurosis, hereditary or acquired macular degeneration, Best disease, retinal detachment, gyrate atrophy, choroideremia, angioid streaks, high myopia (degenerative myopia), idiopathic neovascular maculopathy, pattern dystrophy as well as other dystrophies of the RPE and retinal damage due to any kind of injury.

Retinal pigment epithelium cells, as derived by the methods of this disclosure, may be used in cell transplantation, to generate transplant tissue, or to screen therapeutic compounds or substances that influence the function or metabolism of RPE cells. In one embodiment, RPE cells as derived by the methods of this disclosure may be used for implanting the generated RPE cells into the retina to treat retinal disorders, diseases and conditions. In one embodiment, RPE cells may be implanted in a subject's eye, for example, patients with retinal disorders, diseases and conditions. In one embodiment, the RPE cells may be implanted into animal models of retinal disorders, diseases and conditions.

In one embodiment, RPE cells as derived by the methods of this disclosure may be cryopreserved either as single cells, aggregates or tissues to generate a cell bank for cell transplantation that may be required at multiple clinical sites. RPE cells may be seeded onto a scaffold and cryopreserved to make tissue banks of transplantation-ready tissue. Scaffolds can comprise any materials known in the art and may be maintained during transplantation such as amniotic membranes used in corneal surgery, paralyene or degradable material such as fibrin hydrogel scaffolds, PLGA or porcine-derived collagen scaffolds. The media described in the methods of this disclosure may be used as a recovery media of RPE cells or RPE-seeded scaffolds to prepare for or recover from transplantation.

In one embodiment, RPE cells as derived by the methods of this disclosure may be used for toxicity studies such as phototoxicity, toxicity tests and retinal excitotoxicity and for evaluating efficacy of therapeutic drugs.

In one embodiment, RPE cells derived by the methods of this disclosure may be used for large-scale manufacture, large-scale expansion or automated production of RPE cells while ensuring a high cell purity (e.g. >80%, or >90%, or >95%) and functionality for further downstream applications.

In one embodiment, RPE cells as derived by the methods of this disclosure may be used for gene therapy-related applications focused on treatment of patients with RPE-associated disease mutations or for optimizing gene therapy parameters in vitro prior to animal or clinical studies.

In one embodiment, RPE cells as derived by the methods of this disclosure from patient-derived iPSCs may be used as a diagnosis tool to identify or predict disease causative mutations.

In one embodiment, RPE cells as derived by the methods of this disclosure may serve as therapeutics for eye conditions that include, but are not limited to retinal diseases or disorders related to retinal dysfunction, retinal injury or loss of RPE function. A non-limiting list of conditions that may be treated with the RPE cells of the invention comprises age-related macular degeneration (AMD), glaucoma, cataracts, retinitis pigmentosa (RP), Stargardt disease (SD), Proliferative vitreoretinopathy (PVR), Diabetic retinopathy (DR), lebers congenital amaurosis, hereditary or acquired macular degeneration, Best disease, retinal detachment, gyrate atrophy, choroideremia, angioid streaks, high myopia (degenerative myopia), idiopathic neovascular maculopathy, pattern dystrophy as well as other dystrophies of the RPE and retinal damage due to any kind of injury.

In one embodiment, the RPE cells derived by the methods of this disclosure may be co-cultured with other cell types for various downstream applications. The RPE cells may be co-cultured in vitro or assembled with other retinal cell types such as but not limited to retinal progenitor cells, photoreceptor progenitors or matured photoreceptors (such as rod and cone photoreceptors), whole retina or developing retina, choroidal-like tissue consisting of endothelial cells, fibroblasts or pericytes. Such a co-culture or assembly may mimic the retinal organization in vivo.

In one embodiment, methods of this disclosure may comprise conditioned media derived after culturing the RPE cell precursors, immature RPE or mature RPE in the first, second, third, fourth or maturation media, as described above. Such conditioned media may be used further for isolation/purification of cellular factors, exosomes or the like which may be used further for treatment, prevention or diagnosis of RPE-associated diseases.

In one aspect of this disclosure are provided methods for differentiating PSCs to obtain immature and/or mature RPE. In one embodiment, methods of differentiating PSCs to obtain immature and/or mature RPE may comprise: a) culturing a population of PSC in a first culture environment comprising an extracellular matrix protein and an inhibitor of BMP signaling for between about 0 to 60 hours; b) culturing cells of step a) in a second culture environment comprising an extracellular matrix protein and a second cell culture medium comprising a basal medium and one or more of IGF and an agonist of FGF signaling for between about 0 to 60 hours; c) culturing cells of step b) in a third culture environment comprising an extracellular matrix protein and a third cell culture medium comprising a basal medium and one or more of IGF and an agonist of activin signaling for between about 2 to 5 days; and d) culturing cells of step c) in a fourth culture environment comprising an extracellular matrix protein and a fourth cell culture medium comprising a basal medium and one or more of an agonist of activin signaling, an agonist of wnt signaling, and an antagonist of FGF signaling for between about 5 to 10 days to derive a population of immature RPE.

In one embodiment, methods for differentiating PSCs to obtain immature and/or mature RPE may further comprise dissociating the population of immature retinal pigment epithelial cells and replating the dissociated cells to enrich for immature retinal pigment epithelial cells. In one embodiment, the dissociated population of immature retinal pigment epithelial cells are not enriched through manual dissection, particle-based enrichment, or ligand-mediated enrichment.

In one embodiment, methods for differentiating PSCs to obtain immature and/or mature RPE may further comprise maturing the population of immature retinal pigment epithelial cells for between about 1 to 5 weeks. In one embodiment, the population of immature retinal pigment epithelial cells are matured in contract with a maturation medium comprising a basal medium and at least a soluble source of iron. In one embodiment, the maturation medium further comprises one or more of a steroid hormone and a polyamine.

In one embodiment, components of the methods for differentiating PSCs to obtain immature and/or mature RPE may be as described above.

In another aspect, methods of this disclosure may comprise culturing a population of PSC under non-adherent conditions to produce three-dimensional retinal organoids. Such methods for producing three-dimensional retinal organoids may further comprise differentiating a population of cells fated for the retinal lineage (essentially as described above). Briefly, a population of PSC may be exposed to a first culture environment comprising an extracellular matrix protein and an inhibitor of BMP signaling for between about 0 to 60 hours to derive an arising population of cells (e.g. fated for the retinal linage). Next, the arising population of cells (e.g. fated for the retinal lineage) may be exposed to a second culture environment (essentially as described above) for between about 12 to 60 hours to derive a further arising population of (eye field) progenitors. Next, the further arising population of (eye field) progenitors may be exposed to a third culture environment (essentially as described above) for between about 2 to 5 days.

Cells obtained after having been exposed to a third culture environment (essentially as described above) for between about 2 to 5 days may then be subsequently exposed to a neural retina/RPE induction medium for between 4 to 10 days to form aggregates comprising distinct domains of neural retina and/or RPE. Aggregates comprising distinct domains of neural retina may be detached/dissociated from a culture surface by carving/scratching/etching a grid-like pattern, such as with a pipette tip or the like, through the monolayer to release “sheets” or retinal tissue. The resulting retinal tissue may then be cultured in a retinal organoid formation medium for between about 2 to 6 days under non adherent conditions to form retinal organoids comprising neural retina, which may be further matured in a retinal organoid maturation medium (under non adherent conditions) for between 30 to 200 days.

A neural retina/RPE induction medium may comprise a basal medium (essentially as described above), and one or more of progesterone, putrescine, transferrin and heparin. A neural retina/RPE induction medium may further comprise a member of the bone morphogenetic protein (BMP) family, preferably BMP-4.

A retinal organoid formation medium may comprise a basal medium, as described above, and one or more additional supplements, such as putrescine, insulin or transferrin. A retinal organoid maturation medium may comprise a basal medium, as described above, and one or more additional supplements added at different stages of retinal organoid maturation. Such additional supplements in a retinal organoid maturation medium may comprise serum, a retinoic pathway activator, a retinoic acid receptor activator, a retinoic acid agonist, taurine, lipids or N2 Supplement-A.

A first culture environment, a second culture environment, and/or a third culture environment for generating retinal organoids may comprise nicotinamide. A second culture environment and/or a third culture environment for generating retinal organoids may comprise nicotinamide

In one embodiment, a population of PSC may be seeded into a microwell device. In one embodiment, the population of PSC may be seeded into an uncoated microwell device to obtain 3D spheroids or uniformly sized aggregates of PSCs. Such 3D spheroids may be exposed in sequence, as described above, to a first, second, and third culture environment. In one embodiment, the 3D spheroids or aggregates of PSCs are seeded onto a plate coated with an extracellular matrix protein (e.g., Matrigel, vitronectin or Synthemax (II)) prior to exposure to the first, second, and third culture environment.

A cell population arising after exposure to the third culture environment for between about 2 to 5 days may subsequently be exposed to a neural retina/RPE induction medium (essentially as described above) for between 4 to 10 days to form aggregates comprising distinct domains of neural retina and/or RPE. Once aggregates comprising distinct domains of neural retina and/or RPE are formed, a grid-like pattern may be carved by a pipette tip or the like through the monolayer, and the released “sheets” or retinal tissue may self-organize into structures that eventually develop in suspension into retinal organoids, in appropriate media as described above (and in the absence of an extracellular matrix protein).

In one embodiment, retinal organoids generated by methods of this disclosure comprise retinal progenitors or retinal progenitor cells that generate all cell types of neural retina, as well as cells that differentiate into RPE. Cell types of neural retina may comprise precursors of photoreceptors, retinal progenitor cells, retinal ganglion cells, retinal neurons photoreceptor progenitor cells, rod and cone photoreceptor cells, bipolar cells, amacrine cells, retinal ganglion cells, muller glia cells and horizontal cells.

### Kits

In another aspect of this disclosure are provided media, or kits which may comprise instructions for performing differentiation methods as described herein (e.g step-wise differentiation of progressively more committed precursors into immature RPE and/or mature RPE). The kits described herein may further comprise other materials such as but not limited to supplements, buffers, diluents, solutions and package inserts with instructions for performing any of the methods described herein.

In another aspect of this disclosure are provided media or kits for carrying out methods as contemplated herein, such as to differentiate developmentally earlier precursors into immature RPE (e.g. PSC-derived immature RPE). Said another way, media and kits are provided for carrying out methods of step-wise differentiation of progressively more committed precursors into immature RPE and/or mature RPE.

Such kits may comprise first, second, third and fourth culture media, as described above. Kits may further comprise an extracellular matrix protein, as described above. In one embodiment, the developmentally earlier precursors may comprise a population of cells fated for the retinal lineage, a population of eye field progenitors and/or a population of optic vessel progenitors. In one embodiment, the developmentally earlier precursors may be derived from PSCs as described above. In one embodiment, the first culture medium may comprise an inhibitor of BMP signaling, as described above. In one embodiment, the first culture medium is free of one or both of i) a direct wnt signaling antagonist and ii) an agonist of activin signaling, as described above. In one embodiment, the second cell culture medium comprises a basal medium and one or more of IGF and an agonist of FGF signaling, as described above. In one embodiment, the third cell culture medium comprises a basal medium and one or more of IGF and an agonist of activin signaling, as described above. In one embodiment, the fourth cell culture medium comprises a basal medium and one or more of an agonist of activin signaling, an agonist of wnt signaling, and an antagonist of FGF signaling, as described above. The duration of exposure to the first, second, third and fourth culture media and the concentration of components of the first, second, third and fourth culture media may be as described above.

In another aspect of this disclosure are provided media or kits for differentiating developmentally earlier precursors into mature RPE (e.g. PSC-derived RPE), which may comprise first, second, third, fourth and maturation media, as described above. Kits may further comprise an extracellular matrix protein, as described above. In one embodiment, the maturation media comprises a basal medium and at least a soluble source of iron, as described above. In one embodiment, the maturation media further comprises one or more of a steroid hormone and a polyamine, as described above.

In another aspect of this disclosure are provided media or kits for enriching immature RPE (e.g. PSC-derived RPE), which may comprise first, second, third, fourth and enrichment/replating culture media, as described above. Kits may further comprise an extracellular matrix protein, as described above. In one embodiment, dissociating a population of immature RPE and replating the dissociated cells enriches for immature RPE. In one embodiment, a dissociated population of RPE need not be enriched by manual dissection, particle-based enrichment, or ligand-mediated enrichment. In one embodiment, kits for enriching immature RPE may comprise instructions for dissociating a population of immature RPE and replating the dissociated cells for enriching immature RPE. In one embodiment, the enrichment/replating medium may be serum-free. In one embodiment, the enrichment/replating medium comprises a RHO/ROCK pathway inhibitor or comprises a RHO/ROCK pathway inhibitor added as a supplement, as described above.

In another aspect of this disclosure are provided media or kits for producing three-dimensional retinal organoids (e.g. PSC-derived retinal organoids), which may comprise first, second, and third medi, neural retina/RPE induction media, retinal organoid formation medium and optionally retinal organoid maturation media, all as described above. Kits may further comprise an extracellular matrix protein, as described above. Kits may further comprise a microwell device, as described above.

The following non-limiting examples are illustrative of the present disclosure.

### EXAMPLES

### Example 1: Maintenance and Differentiation of PSCs

Human pluripotent stem cells (PSCs) were maintained in mTeSR™-branded media such as mTeSR™ Plus (STEMCELL Technologies), on either Corning® Matrigel® or on Vitronectin (Thermo Fisher Scientific). PSCs were passaged as described by the manufacturer, but generally cultures were ready for passage when the majority of colonies were large, compact, and had dense multi-layered centers (e.g. approximately 70-80% confluent, and exhibited <10% differentiation). PSCs were split at a ratio of 1/10 to seed for initiating RPE differentiation. Herein, various iPSC lines (WLS-1C, SCTi003-A and STIPS-M001) and ESC lines (H9) were used in downstream differentiation experiments. Whether maintaining or differentiating hPSC, the cells could either be seeded as clumps or as single cells.

### Example 2: Selected Validation Protocols

At different stages of differentiation, but usually day 14 immature RPE and day 49 mature (d) RPE, cells could be analyzed by flow cytometry. Briefly, 0.1×10⁶ RPE were aliquoted per well of a 96 well plate, centrifuged at 500×g for 3 minutes, and the supernatant was removed. The pellet was resuspended in 100 μL of Zombie Violet Fixable Viability Dye (Biolegend) in D-PBS (1/1000 of stock) and incubated in the dark at room temperature for 10 minutes. 100 μL of D-PBS was added to each well, centrifuged at 500×g for 3 minutes, and the supernatant was removed. The pellet was resuspended in 200 μL of 4% PFA and incubated in the dark at room temperature for 15 minutes. The cells were washed in 200 μL of buffer, and the pellet was resuspended in 200 μL of 0.2% Saponin, incubated in the dark at room temperature for 15 minutes, centrifuged at 500×g for 3 minutes, and the supernatant was removed. The cells were resuspended in either 100 μl of anti-PMEL17 (Biolegend AF647, Clone HMB-45) or anti-RPE65 (Novus Biologicals Clone: 401.8B11.3D9) antibody in 0.2% Saponin and incubated in the dark at room temperature for 30-60 minutes. Following incubation, one or two washes with 100 μl or 200 μl of 0.2% Saponin were performed. Depending on the primary antibody used secondary antibody staining may be required. For PMEL17 staining, 200 μl of 0.2% Saponin was added to each well, resuspended and analysed by fluorescence-activated cell sorting (FACS). In the case of, RPE65 staining, 100 μl of secondary donkey anti-mouse antibody (Jackson Immuno, AF647 Fab2) in 0.2% Saponin was added to each well and incubated in the dark at room temperature for 30-60 minutes. Following incubation, one or two washes were performed as described above. Cells were ready for analysis by flow cytometry.

RPE pigmentation levels of cells at different stages of differentiation, but usually day 49 mature (d) RPE, could be analysed by a melanin content assay. To prepare a melanin standard curve, synthetic melanin (Sigma-Aldrich) was reconstituted in 4.26 mM NaOH to prepare a concentration range of 0, 7.825, 15.65, 31.3, 62.5, 125, 250 and 500 μg/mL in triplicates in a 96 well tissue culture plate. A total of 4×10⁵ RPE were added per well of a 96 well u bottom plate and centrifuged at 500×g for 3 minutes. Once the supernatant was removed, cells were resuspended in 100 μL of D-PBS and transferred to a 96 well tissue culture plate. RPE were centrifuged at 300×g for 3 minutes and absorbance was measured at 490 nm. Melanin content was calculated relative to the synthetic standard.

### Example 3: Retinal Pigment Epithelium (RPE) Differentiation

### i) Generation of Cells Fated for the Retinal Lineage and Eye Field Progenitors (Day 0 to 4)

PSC were maintained as described in Example 1, and day 0 cultures (roughly 60-80% confluent) were washed twice with 1 ml of sterile 0.5 mM EDTA before 1 ml of a first differentiation medium (Medium A) was added per well of a 6 well plate. Such medium may alternatively be referred to as a first culture medium, and may comprise an inhibitor of BMP signaling (BMPi⁺) and no inhibitor of Wnt signaling (WNTi⁻) and no Activin A (A⁻). PSCs were then scraped and triturated several times before ^{~}100 μl of clumps, comprising about 1.2×10⁵ cells, were seeded per well of a Matrigel® or Vitronectin coated 12 well plate in 1 ml of Medium A, to initiate differentiation. The plate was rocked to distribute the clumps, and incubated at 5% CO₂ and 37° C. On day 1, the medium was exchanged.

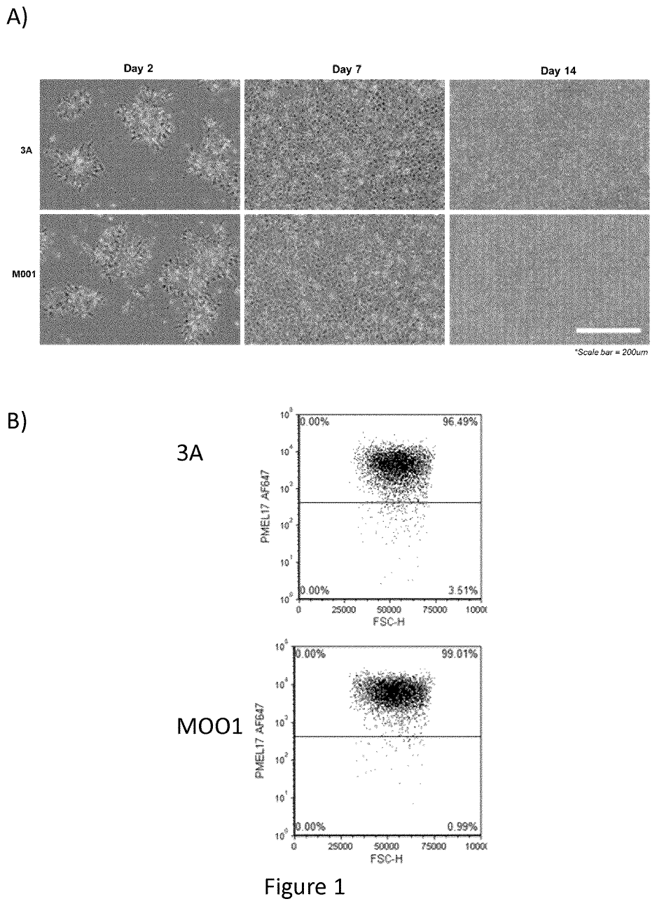

On Day 2, Medium A was removed and 1 ml of a second differentiation medium (Medium B) was added per well. Such may alternatively be referred to as a second culture medium, and may comprise no inhibitor of BMP signaling (BMPi⁻), no inhibitor of Wnt signaling (WNTi⁻), and no Activin A (A). Plates were incubator at 5% CO₂ and 37° C. At this stage cultures should have clumps displaying a rosette like morphology, as shown in FIG. 1A.

### ii) Generation of Optic Vessel Progenitors (Day 4-7)

On day 4, medium was removed and 1 ml of a third differentiation medium (Medium C) was added per well. Such medium may alternatively be referred to as a third culture medium, and may comprise Activin A (A⁺) and no inhibitor of BMP signaling (BMPi⁻) or Wnt signaling (WNTi⁻). Plates were incubated at 5% CO₂ and 37° C. Preferably, cultures should reach ^{~}100% confluence in the next 1-2 days.

On day 6, medium was exchanged, and plates were incubated at 5% CO₂ and 37° C. for an additional day. An exemplary day 7 culture is shown in FIG. 1A.

iii) Generation of Immature Retinal Pigment Epithelial (RPE) Cells (Day 7-14)

On day 7 the medium was replaced with 1 ml of a fourth differentiation medium (Medium D) per well with medium changes on days 9, 11 and 13. Such medium may alternatively be referred to as a fourth culture medium, and may comprise Activin A (A⁺) and a wnt agonist, but no inhibitor of BMP signaling (BMPi⁻) and/or Wnt signaling (WNTi⁻). Plates were incubated at 5% CO₂ and 37° C. An exemplary day 14 uniform monolayer of polygonal immature RPE is shown in FIG. 1A.

### iv) RPE Passaging & Maturation (Day 14, 49)

On day 14, medium was removed, washed twice with 0.5 ml of D-PBS, and contacted with 0.5 mL of a trypsin-based enzymatic solution. Monolayers were detached using a cell lifter, triturated several times, and a single cell suspension comprising immature RPE was transferred to a tube where the volume was brought up to 10 ml in a basal medium comprising progesterone, putrescine, and soluble iron (“+3F”). A filtrate through a 70 μm cell strainer was centrifuged at 300×g for 5 min and the pellet was resuspended in 4 ml of a basal medium +3F (further comprising a RHO/ROCK pathway inhibitor). Cell counts were performed by conventional means, and 1×10⁵ cells/cm² cells were seeded onto plates coated with Matrigel®/Vitronectin (as prepared according to manufacturer's instructions). Prior to seeding, the cells could be analyzed by flow cytometry, as described in Example 2. Maturation may be initiated when approximately 50% or more cells are positive for PMEL17. Cultures with >50% PMEL17 expression at day 14 may reasonably be expected to enrich in the foregoing enrichment passage to >80%, and potentially >90%. Thus, the RPE enrichment described herein does not involve manual dissection, magnetic-activated cell sorting (MACS) or separation methods using magnetic particles.

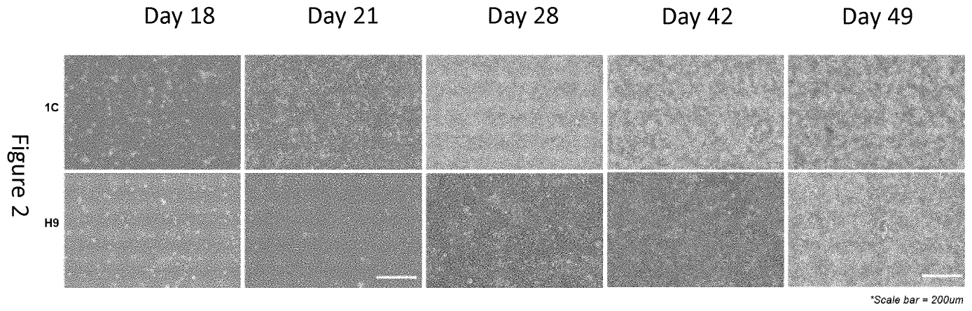

Seeded cells were incubated at 5% CO₂ and 37° C., and 4 mL medium changes were performed on day 16 and 18. On day 21 and thereafter, the cells were cultured in basal medium+3F (e.g. maturation medium) with medium changes every 3-4 days until passaging on day 49. Formation of a cobblestone-like morphology and visible cell-to-cell or tight junctions observed on days 18, 21, 28 and 42 is shown in FIG. 2. On day 49, a uniform monolayer of pigmented RPE with visible tight junctions should be observed as shown in FIG. 2.

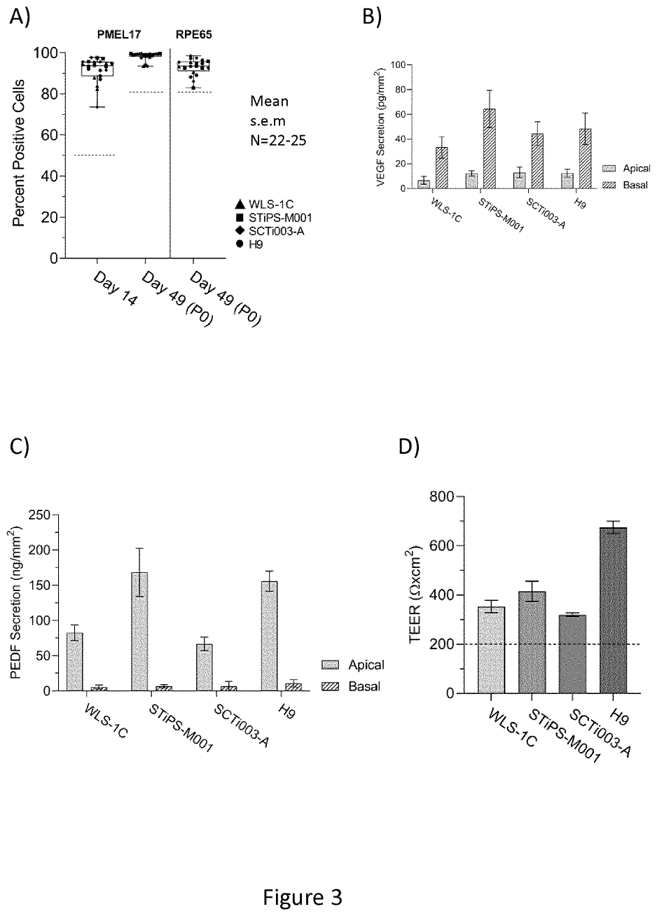

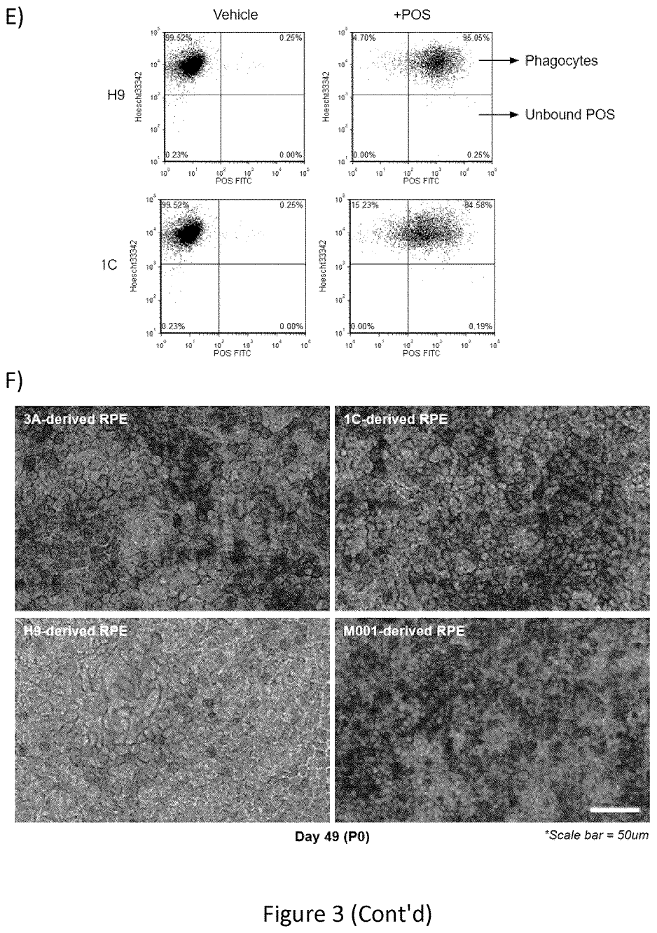

On day 49 the cells were washed twice with 1 ml of D-PBS before 1 ml of a trypsin-based enzymatic solution was added to each well. Monolayers were detached using a cell lifter, triturated to generate a single cell suspension comprising mature RPE, and transferred to a tube in which the volume was brought up to 10 ml in basal medium+3F. A filtrate through a 70 μm cell strainer was centrifuged at 300×g for 5 min and the pellet was resuspended in 4 ml of basal medium+3F. Cell counts were performed by conventional means. 1×10⁵ cells/cm² cells suspended in 4 ml of basal medium +3F were seeded onto plates coated with Matrigel®/Vitronectin (as prepared according to manufacturer's instructions). Prior to seeding, the cell suspension could be analyzed by flow cytometry as described in Example 2. At this stage a majority (e.g., 90-95%) of cells may be positive for PMEL17 and/or RPE65 (FIG. 3).

### Example 4: Characterization of Mature RPE

Four different hPSC lines were differentiated essentially as described in Example 3, and day 49 cells were assessed for maturity through marker expression, growth factor secretion, barrier function, ability to phagocytose photoreceptor outer segments, and melanin production.

Matured RPE cells derived from the 4 different PSC lines were analysed by flow cytometry on day 49 (essentially as described in Example 2) for PMEL17 and RPE65 expression (FIG. 3A). Differentiation efficiencies of 91.4% and 96.6% were observed on Day 14 and Day 49 (PO) respectively, based on PMEL17 marker expression, and 93% of day 49 cells expressed the maturation marker RPE65.

Cells at different stages of differentiation, but usually day 14 immature RPE, could be assessed for the apical and basal secretion of vascular endothelial growth factor (VEGF) and pigment epithelium-derived factor (PEDF) using commercially available VEGF (Thermo Fisher) and PEDF (Abcam) ELISA kits. Briefly, cells were washed twice with 0.5 ml of D-PBS before 0.5 ml of a trypsin-based enzymatic solution was added to each well. Monolayers were detached using a cell lifter, triturated, and a single cell suspension comprising immature RPE was brought up to 10 ml in RPE maturation medium comprising a basal medium, and progesterone, putrescine, and soluble iron (“+3F”), which was passed through a 70 μm cell strainer to filter out RPE tissue or contaminating cell or tissues. The filtrate was resuspended in 4 ml of RPE maturation medium, further comprising an RHO/ROCK pathway inhibitor. Roughly 4×10⁵ cells were seeded onto the apical side of a cell culture insert (0.4 μm, 12 mm PET, Sterlitech) pre-coated with either Corning® Matrigel® or Vitronectin. Roughly 0.5 ml and 1.5 ml of medium was respectively maintained on the apical and basal sides of the insert during incubation at 37° C. and 5% CO₂. Medium from the basal and apical side was changed on Day 16 and 18. On Day 21 and thereafter RPE were maintained in a maturation medium without a RHO/ROCK pathway inhibitor, with changes every 3-4 days until day 46-47. On Day 49 or 50, 20 μl of conditioned medium was collected from the apical and basal chambers and analyzed by ELISA essentially in accordance with the manufacturer's instructions.

PEDF ELISA was carried out in a similar way as per the VEGF ELISA protocol described above, except for some modifications. On Day 49 or 50, 10 μL of conditioned medium was collected from the apical and basal chambers and conditioned medium was diluted 100× in RPE maturation medium and further diluted in sample diluent buffer to achieve a final dilution of 2000×. Thereafter samples were run according to the manufacturer's instructions, with slight modifications. The results showed that mature RPE exhibit an increased basal secretion of VEGF for all cell lines tested (FIG. 3B), and an increased apical secretion of PEDF for all cell lines tested (FIG. 3C).

Cells at different stages of differentiation, but usually day 14 immature RPE, could be assessed for barrier function by measuring transepithelial electrical resistance (TEER). Briefly, hPSCs from 4 different cell lines were differentiated to immature RPE (Day 14), as described in Example 3, washed twice with 0.5 ml of D-PBS, and contacted with 0.5 ml of a trypsin-based enzymatic solution. Monolayers were detached using a cell lifter, triturated, and a single cell suspension comprising immature RPE was resuspended in 10 ml of RPE maturation medium before being passed through a 70 μm cell strainer and further centrifuged. Cell pellets were resuspended in 4 ml of RPE Maturation medium comprising a RHO/ROCK pathway inhibitor, and roughly 4×10⁵ cells were seeded onto the apical side of a cell culture insert (0.4 μm, 12 mm PET, Sterlitech) pre-coated with Corning® Matrigel®. One blank cell culture insert coated with Corning® Matrigel® was used as a control. Roughly 0.5 ml and 1.5 ml of medium was maintained in the apical and basal chambers during incubation at 37° C. and 5% CO₂, with medium changes on Day 16 and 18. On Day 21 and thereafter RPE were maintained in RPE Maturation Medium without the RHO/ROCK pathway inhibitor, with changes every 3-4 days until day 46-47. Cells grown on the inserts sat at room temperature for 10-15 minutes before measuring TEER. An Epithelial Voltohmmeter EVOM2™ equipped with chopstick electrode (World Precision Instruments) sterilized in 70% ethanol was used to measure TEER values of Day 49 differentiated cells. The electrodes were submerged into the apical and basal chambers and electrical measurements were recorded for each well. TEER values were subtracted from the blank control and multiplied by the insert area factor. Mature RPE cells from all 4 hPSC lines tested exhibited TEER values greater than 200 Ω×cm² (FIG. 3D). These results suggest that day 49 cells form tight junctions and sufficient barrier to allow for polarization and polarized secretion of factors such as PEDF and VEGF. Barrier function could be maintained for an additional 4 weeks (data not shown).

Cells at different stages of differentiation, but usually mature (d) (at least day 77) RPE, could be assessed for the ability to phagocytose photoreceptor outer segments (POS). POS obtained from bovine eyes (InVision BioResources) were conjugated with fluorescein-5-isothiocyanate (FITC). Briefly, 10 mg of FITC Isomer I was resuspended in 0.1M sodium bicarbonate buffer and incubated at room temperature on a rotating platform for 1 hour, protected from light. Thereafter, non-resuspended FITC solids were centrifuged at ≥3000 g, and the pellets were resuspended in 5 ml of DMEM/F12 medium. The FITC Isomer I solution was slowly added to ^{~}200×10⁶ POS while swirling the POS, and the combination was incubated for 1.5 hours at room temperature on a rotating platform, protected from light. A pellet of labelled POS was resuspended in 1.5 ml of DMEM/F12, pelleted again to wash out any unconjugated FITC Isomer I, and repeated. FITC conjugated POS was resuspended to a final volume of 5 ml of DMEM/F12 and 12×10⁶ POS/vial aliquots were prepared before centrifuging the vials at ≥3000 g before freezing the pellets −80° C. For the RPE POS assay, day 14 immature RPE were matured until at least day 49 in either tissue culture plates or on 12 mm cell culture inserts. In 12-well tissue culture plates, ^{~}1×10⁵ cells/cm² were seeded per well pre-coated with Corning® Matrigel® and incubated at 37° C. and 5% CO₂ with 2 mL Maturation Medium changes every 3-4 days until passaging on Day 49. On day 49, frozen FITC-POS vials were thawed on ice and 10×10⁶ FITC-POS were added per 2 ml of RPE maturation medium. Untreated cells without the addition of POS or cells containing FITC-POS in the presence of 50 μg/mL anti-αvβ5 were used to obtain baseline fluorescence. After 16 hours of incubation the medium was removed, and wells were rinsed 3-5 times with 2 ml of D-PBS to remove any unbound FITC-POS. 0.5 mL of a trypsin-based enzymatic solution was added to each well and incubated at 37° C. for 10 min. Monolayers were detached using a cell lifter, triturated, and a single cell suspension was resuspended in 10 ml of RPE maturation medium before being passed through a 70 μm cell strainer and further centrifuged. Cell pellets were resuspended in 0.4% Trypan blue in D-PBS to quench bound FITC-POS, the volume was made up to 10 mL in RPE maturation medium and centrifuged at 300×g for 5 minutes. Pellets were resuspended in 1 ml of buffer with 1:1000 Hoechst 33342 before performing flow cytometry as described in Example 2 to quantify FITC-POS Digestion.

The data show that only with the addition of FITC-conjugated POS do the cells stain positive for FITC (FIG. 3E). Untreated cells without the addition of POS (Vehicle) show a majority of 99.52% non-phagocyte cells that have not internalized POS. A very low population of cells (0.25% & 0.19% in H9 and 1C-derived RPE respectively) displayed POS that attached to the cells but were released by proteolysis, referred to as “unbound POS”. Overall, the data suggest that a majority of the 1C and H9-derived RPE are able to phagocytose POS.

Matured RPE cells derived from the 4 different PSC lines were imaged on Day 49 (PO). Representative microscopy images show appreciable pigmentation levels among cells differentiated from each hPSC cell line (FIG. 3F).

### Example 5: Effect of Culture Medium Composition on RPE Differentiation, Purity, and Pigmentation

Cells were cultured and differentiated essentially as described in Example 3, except medium composition differences were explored, including i) cytokine and small molecule concentrations in the third and fourth culture media, and ii) different basal media for RPE maturation.

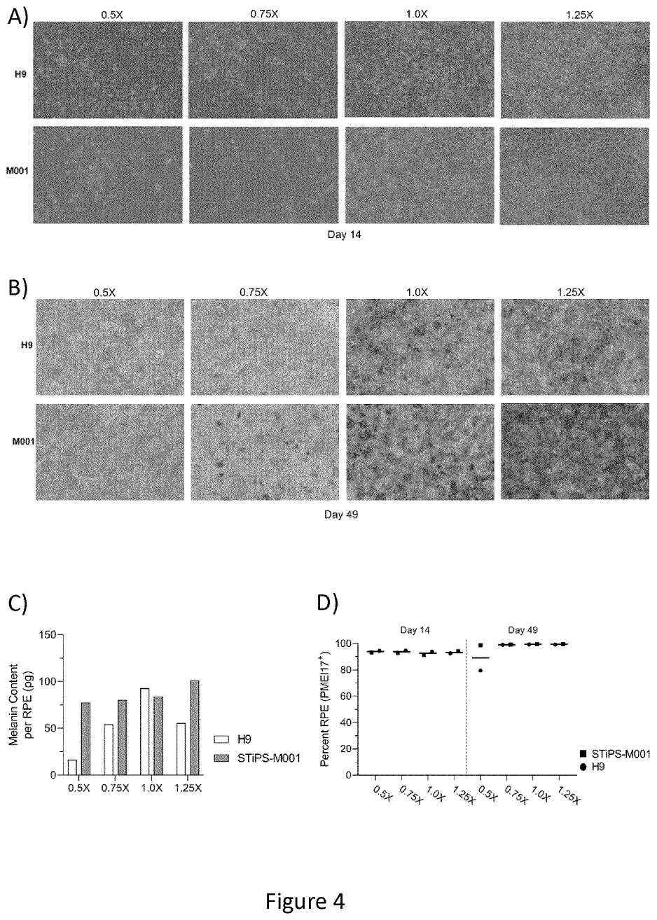

The concentration of each of the cytokines and small molecules present in the third and fourth culture media, as used in Example 3, was varied to assess the effects on differentiation of immature and mature RPE. The concentrations in the third medium were tested at 0.5×, 0.75×, 1×, and 1.25×, and the concentrations in the fourth medium were matched at 0.5×, 0.75×, 1×, and 1.25×. The results show similar efficiency of immature RPE generation in both H9 and M001 for all conditions tested (FIG. 4A). However, at lower concentrations of cytokines and small molecules (e.g., 0.5× and 0.75×) RPE pigmentation levels appeared reduced (FIG. 4B), as further confirmed by a melanin content assay performed as described in Example 2 (FIG. 4C). The results also show comparable RPE purity as assessed by PMEL17 flow cytometry among differentiated H9 and M001 for all conditions tested (FIG. 4D).

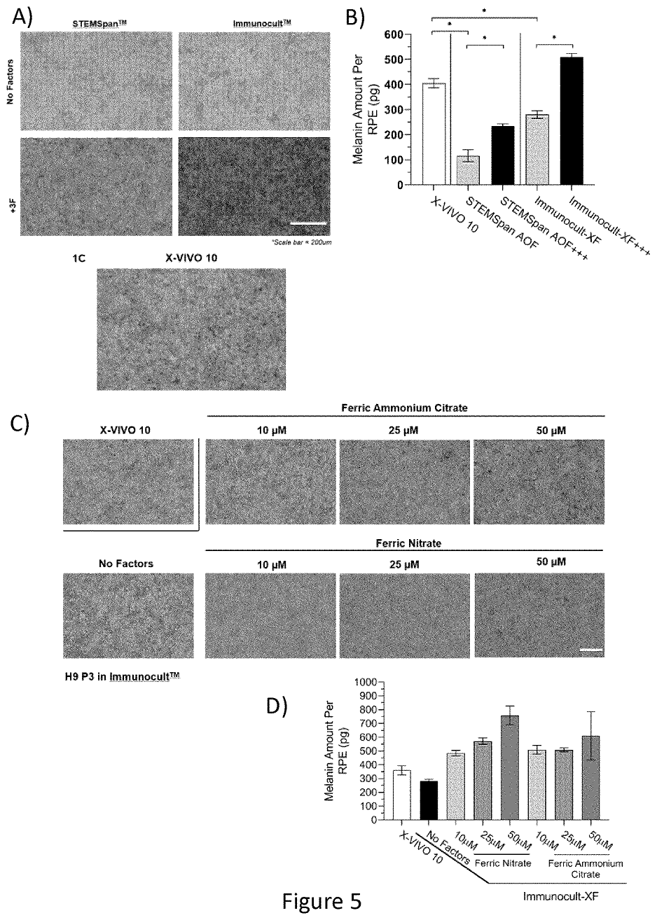

To test the impact of different basal media during the maturation stage, day 14 WLS-1C-derived immature RPE were further cultured essentially as described in Example 3 in three different basal media: X-VIVO 10 (Lonza) as a control; and two different analogous basal medium manufactured by STEMCELL Technologies (StemSpan™- and ImmunoCult™-branded basal media). In comparison to X-VIVO-10, cells matured in STEMCELL-branded media appeared to exhibit reduced pigmentation. However, pigmentation was restored among cells matured in STEMCELL-branded media when supplemented with progesterone, putrescine and a source of soluble iron (e.g. ferric ammonium citrate or ferric nitrate) (FIG. 5A). These findings were further confirmed by a melanin content assay (FIG. 5B). Further, for both ferric nitrate and ferric ammonium citrate, increasing levels of melanogenesis could be induced with increasing concentrations (10 μM, 25 UM and 50 μM) of both iron sources, as observed through brightfield microscopy and a melanin content assay (FIG. 5C and D). The results show that supplementation of basal media as described above can yield greater levels of melanin formation than in a control medium.

### Example 6: High Differentiation Efficiency of Clump and Single-Cell Passaged hPSCs

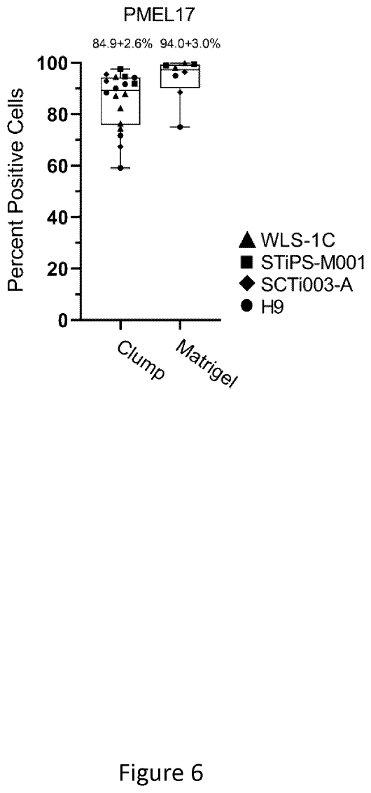

PSCs derived from 4 cell lines 1C, M001, 3A and H9 were passaged either as single cells seeded at 25,000/cm² or as clumps (1/10 seeding ratio) and differentiated under conditions as described in Example 3. Differentiation was assessed by flow cytometry analysis of PMEL17 marker expression (FIG. 6). Both conditions resulted in marked levels of PMEL17 expression, with a slightly higher efficiency observed among single cell passaged cells (^{~}94%) as compared to cells differentiated from clump passaged PSC.

### Example 7: A 3-Stage RPE Differentiation Protocol

As an alternative to the 4-stage differentiation protocol described in Example 3, RPE differentiation was carried out in 3 stages. In the first stage, from day 0-4, hPSCs were differentiated in a medium which included a BMP pathway inhibitor (Noggin) and a wnt pathway inhibitor (DKK1). In the second stage, from day 4-8, cells were cultured in a medium containing Activin-A. In the third stage, from day 8 to 14, cells were cultured in a medium containing activin A, an FGFR-1 inhibitor and a wnt agonist.

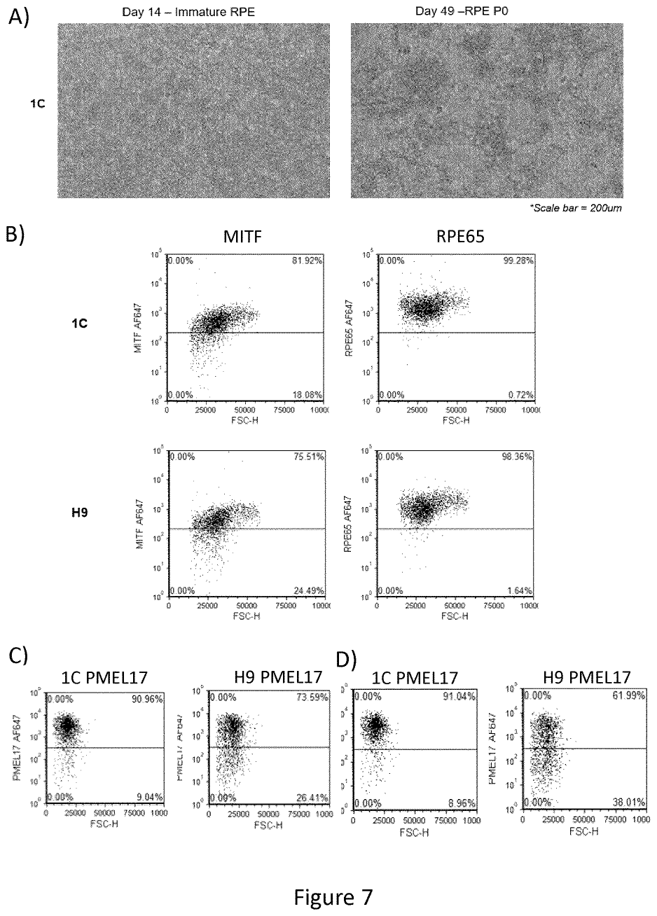

Differentiated cells were analysed by microscopy and flow cytometry for RPE65 and MITF expression. Immature (day 14) and matured (day 49) RPE exhibited polygonal morphology (FIG. 7A). Further, flow cytometry analysis of day 49 cells quantified the expression of MITF (an optic vesicle and RPE marker) and RPE65 (RPE maturation marker) in WLS-1C and H9-derived RPE. The results showed that 1C-derived RPE cells had 99.28% RPE65 positive cells and 81.92% MITF-positive cells, whereas H9-derived RPE cells had 98.36% RPE65 positive cells and 75.51% MITF-positive cells (FIG. 7B). The results suggest that high differentiation and RPE maturation efficiencies were achieved with the 3-stage differentiation protocol. However, when assessing PMEL17 expression between day 14 WLS-1C-derived cells and H9-derived cells, greater variability was observed in the 3-stage differentiation protocol in comparison to the 4-stage protocol of Example 3 (FIG. 7C).

Interestingly, the combination of inhibitors of wnt signaling and of BMP signaling pathways in the culture medium from day 0-4 appeared to result in higher cell death at day 8 (data not shown). When the BMP pathway inhibitor (Noggin) and the wnt pathway inhibitor (DKK1) were replaced by an alternate BMP pathway inhibitor, LDN-193189 and an alternate wnt pathway inhibitor, IWP2, RPE differentiation efficiencies as assessed by PMEL17 marker expression were found to be lower in H9-derived RPE. Flow cytometry analysis of PMEL17 expression showed that H9-derived cells yielded 61.99% PMEL17 positive cells compared to 91.04% PMEL17 positive cells in 1C-derived RPE (FIG. 7D).

### Example 8: a 5-Stage RPE Differentiation Protocol

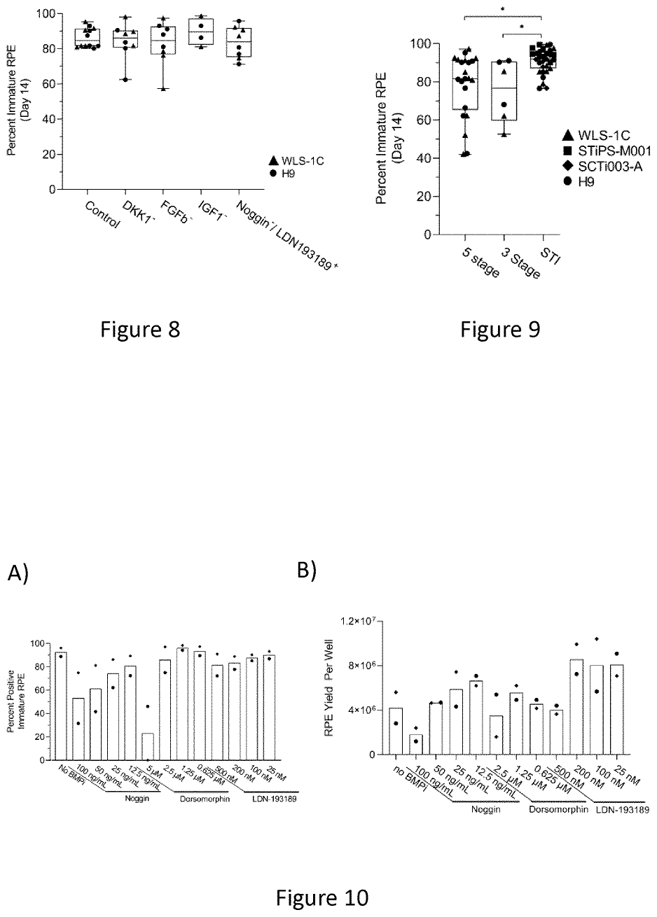

As an alternative to the 4-stage and 3-stage differentiation protocols described in Examples 3 and 7 respectively, RPE differentiation was carried out in 5 stages. In the first stage, from day 0-2, hPSCs were differentiated in a medium comprising 50 ng/ml of Noggin, DKK-1, and IGF-1. In the second stage, from day 2-4, cells were cultured in a medium containing 10 ng/ml of Noggin, DKK1, IGF-1 and FGFb. In the third stage, from day 4 to 6, cells were cultured in a medium containing activin A, DKK1, and IGF-1. In the fourth stage, from day 6 to 8, cells were cultured in a medium containing activin A and an FGFR-1 inhibitor. In the fifth stage, from day 8 to 14, cells were cultured in a medium containing activin A, an FGFR-1 inhibitor and an agonist of wnt signaling. Individual medium components were either removed or replaced with alternate factors and the effect on RPE differentiation was assessed by flow cytometry assessment of 1C and H9-derived percent immature RPE on Day 14. The following conditions were tested: (i) Control with all the medium components of the 5-stage differentiation protocol described above (ii) Removal of DKK1 (iii) Removal of FGFb (iv) Removal of IGF-1 (v) Replacement of Noggin with LDN-193189. The results (FIG. 8) showed that removal of DKK1 or IGF-1 did not have a negative impact on RPE differentiation as assessed by flow cytometry of day 14 percent immature RPE. The results suggested that the RPE differentiation could be achieved in the absence of DKK1.

### Example 9: Comparing Differentiation Efficiencies of the 3 Differentiation Protocols

The efficiencies of the 5-stage differentiation protocol of Example 8, the 3-stage differentiation protocol of Example 7, and the 4-stage differentiation protocol of Example 3 were compared to one another. Flow cytometry analysis of PMEL17 expression among day 14 immature RPE is summarized in the box and whisker plot in FIG. 9. While each protocol demonstrated averages of about 80% or greater, the 4-stage differentiation protocol exhibited the highest efficiency and was the most robust (FIG. 9).

### Example 10: Effect of BMP Inhibition on RPE Differentiation Efficiency

Cells were cultured and differentiated essentially as described in Example 3, but the first culture medium was modified to include different types and concentrations of inhibitors of BMP signaling: (i) no BMP inhibition; (ii) Noggin (12.5 ng/ml, 25 ng/ml & 100 ng/ml); (iii) Dorsomorphin (0.625 UM, 1.25 UM, 2.5 μM & 5 μM); and (iv) LDN-193189 (25 nM, 100 nM, 200 nM & 500 nM).

Percent positive day 14 immature RPE cells from 3A and H9 cell lines were analysed by flow cytometry for PMEL17 expression (FIG. 10A). In addition, immature RPE yield per well was calculated using a hemocytometer (FIG. 10B). The results generally showed improved RPE differentiation at lower concentrations for all the BMP inhibitors tested (FIG. 10A). Specifically, 0.625 UM or 1.25 μM dorsomorphin, and 25 nM or 100 nM LDN-193189 generated the most consistent RPE differentiation efficiency (e.g. >80%) between the tested hPSC cell lines. While a lack of any inhibitor of BMP signaling was observed to yield a high percentage PMEL17+ day 14 immature RPE, its absence appeared to correlate with increased corneal contamination, such as during maturation (data not shown).

### Example 11: Generation and Characterization of Retinal Organoids

PSC maintained essentially as described in Example 1, were formed into aggregates in an Aggrewell™ 800 (STEMCELL Technologies) 24-well plate in mTeSR™1 or mTeSR™ Plus, in accordance with the manufacturer's protocol. Aggregates were recovered from AggreWell™ plates, and plated in Matrigel™ coated plates and cultured up until day 6 essentially as described in Example 3. Thereafter, the cells were cultured in a neural retina/RPE induction medium comprising BMP4 for 3 days, followed by 5 days of culture in the medium lacking BMP4 and comprising one or more of progesterone, putrescine, transferrin and heparin to form aggregates comprising distinct domains of neural retina and/or RPE. Next, aggregates comprising distinct domains of neural retina were dissociated by carving a grid-like pattern using a pipette tip or the like through the monolayer, and releasing sheets of retinal tissue. The retinal-like tissues were then cultured in a retinal organoid formation medium for 4 days from day 14 to day 18 under non adherent conditions to form retinal organoids comprising neural retina. Next, retinal organoids were matured in a retinal organoid maturation medium from day 18 to day 150.

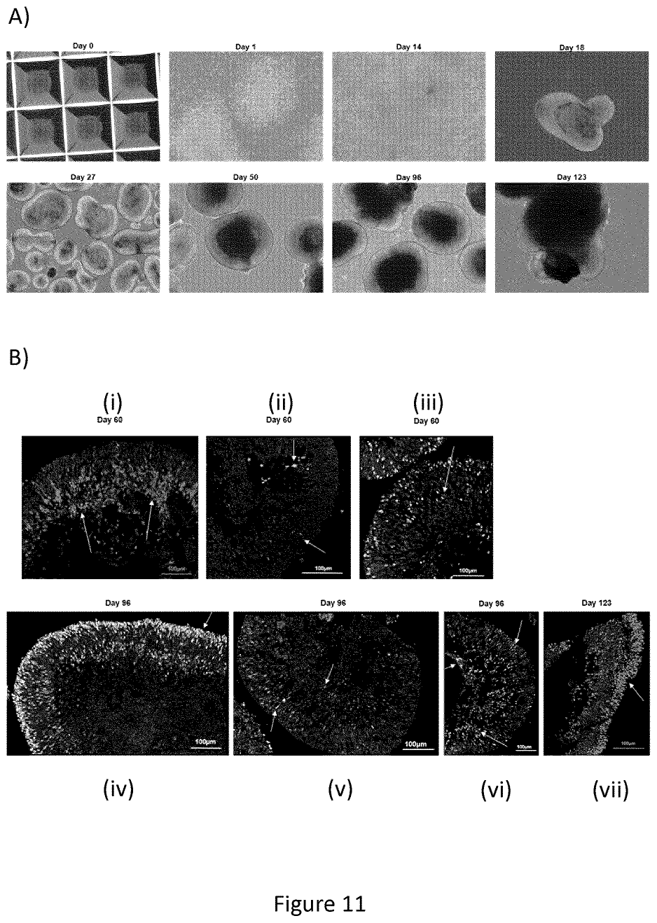

FIG. 11A shows the retinal organoid generation at different stages of development during the above mentioned protocol, including: uniform day-0 PSC aggregates formed in AggreWell™800; day-1 differentiating aggregates adhered to the plate; day-14 cultures comprising distinct domains of neural retina and RPE; day-18 retinal structures with phase bright borders of neural retina; day-27 retinal organoids; and maturation of retinal organoids at days 50, 96, and 123.

FIG. 11B shows the immunohistochemistry staining to characterize cells of the retinal organoids at various stages. Expected markers of retinal organoids were observed at specific stages of the protocol. Day 60 staining data showed that retinal ganglion cells (RGCs) stained with SNCG antibodies localized correctly within the Retinal organoids. RGCs were observed to make cell projections into the neuroblastic layer stained with VSX2 antibodies (i). The generated retinal organoids were further shown to comprise amacrine cells (AP2a) and horizontal cells (Prox1), with amacrine cells organized more towards the RGC layer (ii). The generated retinal organoids were also shown to comprise differentiating retinal progenitor cells (RPCs) and peripherally organized photoreceptor progenitors (PRPs) by staining with OTX2 and CRX antibodies (iii). Day 96 staining with OTX2 and CRX antibodies further showed that photoreceptors increase in density along the periphery of retinal organoids and populate the developing outer nuclear layer (iv). Day 96 staining data also showed that the generated retinal organoids contain amacrine cells (AP2α) and horizontal cells (Prox1), with amacrine cells organized more towards the RGC layer (v). RGC nerve fibre like bundle was also observed on day 96 (Prox1 and AP2α) (vi). Similar organization of retinal organoids containing amacrine cells and horizontal cells was also observed on Day 123 (vii).

1. A method of differentiating a population of cells fated for the retinal lineage, the method comprising:
a) providing a population of pluripotent stem cells (PSC);
b) exposing the population of PSC to a first culture environment comprising an extracellular matrix protein and an inhibitor of bone morphogenetic protein (BMP) signaling in a first cell culture medium;
c) culturing the population of PSC in the first culture environment for between about 0 to 60 hours; and
d) deriving the population of cells fated for the retinal linage.

2. The method of claim 1, wherein the first culture environment is free of one or both of i) a direct wnt signaling antagonist, and ii) an agonist of activin signaling.

3. The method of claim 1, wherein the extracellular matrix protein is coated on a culture surface, and the extracellular matrix protein is vitronectin, laminin, fibronectin, collagen, or a mixture comprising more than one of the foregoing.

4. The method of claim 1, wherein a concentration of the inhibitor of BMP signaling is about 100 nM or lower.

5. (canceled)

6. The method of claim 1 further comprising:
e) exposing the population of cells fated for the retinal lineage to a second culture environment;
f) culturing the population of cells for between about 0 to 60 hours; and
g) deriving a population of eye field progenitors,
wherein the second culture environment comprises the extracellular matrix protein and a second cell culture medium.

7. (canceled)

8. The method of claim 6, wherein the second cell culture medium comprises a basal medium and one or more of insulin-like growth factor (IGF) and an agonist of fibroblast growth factor (FGF) signaling.

9. The method of claim 1, further comprising:
e) exposing the population of cells fated for the retinal lineage to a second culture environment;
f) culturing the population of cells for between about 0 to 60 hours;
g) deriving a population of eye field progenitors;
h) exposing the population of eye field progenitors to a third culture environment;
i) culturing the population of eye field progenitors for between about 2 to 5 days; and
j) deriving a population of optic vessel progenitors,
wherein the second culture environment comprises the extracellular matrix protein and a second cell culture medium and wherein the third culture environment comprises the extracellular matrix protein and a third cell culture medium.

10. (canceled)

11. The method of claim 9, wherein the third cell culture medium comprises a basal medium and one or more of IGF and an agonist of activin signaling.

12. The method of claim 9, wherein an optic vessel progenitor is bipotent for retinal pigment epithelium or neural retina.

13. The method of claim 9, further comprising:
k) exposing the population of optic vessel progenitors to a fourth culture environment;
l) culturing the population of optic vessel progenitors for between about 5 to 10 days; and
m) deriving a population of immature retinal pigment epithelial cells,
wherein the fourth culture environment comprises the extracellular matrix protein and a fourth cell culture medium.

14. (canceled)

15. The method of claim 13, wherein the fourth cell culture medium comprises a basal medium and one or more of an agonist of activin signaling, an agonist of wnt signaling, and an antagonist of FGF signaling.

16. The method of claim 13, further comprising dissociating the population of immature retinal pigment epithelial cells and replating the dissociated cells to enrich for immature retinal pigment epithelial cells.

17. The method of claim 16, wherein the dissociated population of immature retinal pigment epithelial cells are not enriched through manual dissection, particle-based enrichment, or ligand-mediated enrichment.

18. The method of claim 13, wherein about 50% or more of the population of immature retinal pigment epithelial cells express PMEL17.

19. The method of claim 13, further comprising maturing the population of immature retinal pigment epithelial cells for between about 1 to 5 weeks in contact with a maturation medium comprising a basal medium and at least a soluble source of iron.

20. (canceled)

21. The method of claim 19, wherein the maturation medium further comprises one or more of a steroid hormone and a polyamine.

22. The method of claim 19, wherein about 80% or more of the matured retinal pigment epithelial cells (RPE) express RPE65.

23. The method of claim 13, wherein one or more of the first culture environment, the second culture environment, the third culture environment, and the fourth culture environment is serum-free and/or animal component free.

24. The method of claim 13, wherein;
a) one or more of the second cell culture medium, the third cell culture medium, and the fourth cell culture medium lacks an exogenously added inhibitor of BMP signaling; and/or
b) one or more of the first cell culture medium, the second cell culture medium, the third cell culture medium, and the fourth cell culture medium lacks nicotinamide.

25. (canceled)

26. The method of claim 1, wherein the population of PSC are cultured under non-adherent conditions to produce three-dimensional retinal organoids.

27. (canceled)
